(19) United States
(12) Patent Application Publication
(10) Pub. No.: US 2026/0144571 A1
(43) Pub. Date: May 28, 2026

(54) TREATMENT ASSEMBLY SYSTEMS AND METHODS

(71) Applicant: W. L. Gore & Associates, Inc., Newark, DE (US)

(72) Inventors: Jérôme S. Conia, Phoenix, AZ (US); Bryan S. Reep, Detroit Lakes, MN (US)

(21) Appl. No.: 19/382,987

(22) Filed: Nov. 7, 2025

Related U.S. Application Data
(60) Provisional application No. 63/724,070, filed on Nov. 22, 2024.

(51) Int. Cl. A61B 17/34; A61B 17/22

(52) U.S. Cl. CPC A61B 17/3423; A61B 17/3462; A61B 2017/22072; A61B 2217/007

(57) ABSTRACT

Various concepts relate to treatment system including a treatment assembly and an introducer assembly. The treatment assembly may include a treatment chamber having a first end and a second end and defining a chamber lumen therebetween, a valve coupled at the first end of the treatment chamber operable to seal the chamber lumen at the first end, and a coupling projection at the second end of the treatment chamber. The introducer assembly may include an introducer valve having an inner tubular member and the coupling projection is receivable in the inner tubular member such that the coupling projection is both sealed to the inner tubular member and the inner tubular member remains operable to be transitioned between a sealed state and an unsealed state.

### CROSS-REFERENCE TO RELATED APPLICATION

This application claims the benefit of Provisional Application No. 63/724,070, filed Nov. 22, 2024, which is incorporated herein by reference in its entirety for all purposes.

### BACKGROUND

Endoluminal devices are commonly delivered into the body of a patient (e.g., into the patient's vasculature) using an introducer system. Introducer systems typically include valves or similar features to stop backflow of body fluids (e.g., blood) through the introducer while permitting an endoluminal device to pass through the introducer and into the patient's body.

### SUMMARY

Various examples and methods relate to treatment systems used for retrieval of material from a body lumen of a patient. In some examples, the treatment system may assist with retrieval and removal of a device, device components, or fragments of the device from a patient's cardiovascular space, endovascular space, and/or other body lumens. The treatment system may assist with retrieval and removal of biological tissues, biological masses, or fragments of a patient's biological tissues or masses (e.g., tumors, growths, thrombus, diseased tissues). The treatment system may be operable to isolate the removed material from the bodily environment of the patient during a surgical procedure. The treatment systems may include a treatment assembly and an introducer assembly. The treatment assembly may include a treatment chamber, a valve coupled to a first end of the treatment chamber, and a coupling projection on the second end of the treatment chamber. The introducer assembly may include an introducer valve having an inner member configured to receive the coupling projection therein to couple the treatment assembly to the introducer assembly, where the inner tubular member remains operable to be transitioned between a sealed state and an unsealed state when the coupling projection is received therein. The treatment assembly and/or introducer assembly may include a single valve or multiple valves in various examples. In some embodiments, an endoluminal device or tool may be inserted through both an introducer assembly valve and a treatment assembly valve of the treatment system. And, those valves may each be sealed about the endoluminal device or tool such that one or both valves form a hemostatic seal with the endoluminal device or tool. The treatment system can also permit devices to be placed into a vasculature of a patient without exposing the devices to mechanical stress. The treatment assembly and/or the introducer assembly allows for insertion of delivery systems and/or devices therein, which can include fragile components, by limiting or circumventing exposure to mechanical stresses imparted by valve assemblies. This, in turn, can help reduce or prevent damage to delivery systems and/or devices, and components thereof, during both insertion to the vasculature and when loading the delivery system and/or device within the treatment system.

According to one example (“Example 1”), a treatment system includes a treatment assembly and an introducer assembly. The treatment assembly includes a treatment chamber having a first end and a second end and defining a chamber lumen therebetween, a valve coupled at the first end of the treatment chamber operable to seal the chamber lumen at the first end, and a coupling projection at the second end of the treatment chamber. The introducer assembly includes an introducer valve having an inner tubular member where the coupling projection is receivable in the inner tubular member such that the coupling projection is both sealed to the inner tubular member and the inner tubular member remains operable to be transitioned between a sealed state and an unsealed state.

According to another embodiment (“Example 2”), further to Example 1, the introducer valve is operable to seal the second end of the chamber lumen with the coupling projection received in the inner tubular member.

According to another embodiment (“Example 3”), further to any of Examples 1-2, the treatment assembly includes a collar associated with the coupling projection, the collar operable to engage the introducer valve to limit a length that the coupling projection is inserted into the inner tubular member.

According to another embodiment (“Example 4”), further to any of Examples 1-3, the treatment assembly includes a gasket associated with the coupling projection, the gasket being operable to seal to the inner tubular member with the coupling projection inserted into the inner tubular member.

According to another embodiment (“Example 5”), further to Example 4, the gasket is inflatable to seal between the inner tubular member and the coupling projection.

According to another embodiment (“Example 6”), further to any of Examples 1-5, the treatment chamber is longitudinally collapsible and/or diametrically collapsible.

According to another embodiment (“Example 7”), further to Example 6, the treatment chamber includes a plurality of pleats to facilitate longitudinal and/or diametric collapse of the treatment chamber.

According to another embodiment (“Example 8”), further to any of Examples 1-7, at least a portion of the treatment chamber is at least translucent, and optionally transparent.

According to another embodiment (“Example 9”), further to Example 8, a flush port is coupled to the treatment chamber within the portion of the treatment chamber that is at least translucent.

According to another embodiment (“Example 10”), further to any of Examples 1-9, the introducer assembly includes an introducer sheath operable to be in fluid communication with to the introducer valve.

According to another embodiment (“Example 11”), further to any of Examples 1-10, the treatment system further includes a flush port coupled to the treatment chamber, wherein the flush port is in fluid communication with the chamber lumen.

According to another embodiment (“Example 12”), further to Example 11, the flush port is fluidly coupled to a reservoir containing a fluid such that the flush port is operable to move fluid from the reservoir to the chamber lumen.

According to another embodiment (“Example 13”), further to any of Examples 1-12, the treatment system further includes a recirculation loop fluidly coupled to the treatment chamber such that fluid passing into the treatment chamber is directed out of the treatment chamber through the recirculation loop.

According to another embodiment (“Example 14”), further to any of Examples 1-13, the recirculation loop further includes a filter.

According to another embodiment (“Example 15”), further to any of Examples 1-14, the coupling projection is configured to extend into the inner tubular member for less than a full length of the inner tubular member, optionally 50% or less of the full length, optionally 25% or less of the full length, optionally 10% or less of the full length.

According to another embodiment (“Example 16”), further to any of Examples 1-15, the coupling projection has a length that is less than a length of the inner tubular member, optionally 50% or less of the length of the inner tubular member, optionally 25% or less of the length of the inner tubular member, optionally 10% or less of the length of the inner tubular member.

According to another embodiment (“Example 17”), further to any of Examples 1-16, the coupling projection includes one or more visual markings demarcating a desired insertion position of the coupling projection into the introducer valve.

According to another embodiment (“Example 18”), further to any of Examples 1-17, the treatment system further includes a collar associated with the second end of the treatment chamber, the collar including a mating feature operable to mate with a complementary feature of the introducer assembly such that the treatment assembly is longitudinally fixed relative to the introducer assembly.

According to another embodiment (“Example 19”), further to Example 18, the mating feature is rotatable to mate with the complementary feature of the introducer assembly.

According to one embodiment (“Example 20”), a treatment assembly is operable to seal to an introducer valve of an introducer assembly. The treatment assembly includes a treatment chamber having a first end, a second end, and defining a chamber lumen therebetween, a valve coupled at the first end of the treatment chamber operable to seal the chamber lumen at the first end, and a coupling projection at the second end of the treatment chamber, the coupling projection being operable to be inserted into the introducer valve such that the introducer valve is sealable to the coupling projection while being operable to seal and unseal an inner lumen of the introducer valve.

According to another embodiment (“Example 21”), further to Example 20, the treatment assembly further includes a coupling collar associated with the second end of the treatment chamber, the coupling collar including a mating feature operable to mate with a complementary feature of the introducer assembly such that the treatment assembly is longitudinally fixed relative to the introducer assembly.

According to another embodiment (“Example 22”), further to Example 21, the mating feature is rotatable to mate with the complementary feature of the introducer assembly.

According to another embodiment (“Example 23”), further to Example 22, the treatment assembly further includes a gasket associated with the coupling projection.

According to another embodiment (“Example 24”), further to Example 23, the gasket is operable to inflate.

According to another embodiment (“Example 25”), further to any of Examples 20-24, the treatment assembly includes a collar associated with the coupling projection, the collar operable to engage the introducer valve to limit an insertion distance that the coupling projection is inserted into the inner tubular member.

According to another embodiment (“Example 26”), a treatment assembly operable to seal to an introducer valve of an introducer assembly. The treatment assembly includes a treatment chamber having a first end, a second end, and defining a chamber lumen therebetween, a first valve at the first end of the treatment chamber operable to seal the chamber lumen at the first end, a second valve at the second end of the treatment chamber operable to seal the chamber lumen at the second end, and a coupling projection extending from the second valve, the coupling projection being operable to be inserted into the second valve such that the second valve is sealable to the coupling projection while being operable to seal and unseal an inner lumen of the second valve.

According to another embodiment (“Example 27”), a method of treatment includes delivering an introducer assembly into a body lumen of a patient, the introducer assembly including an introducer sheath extending into the body lumen and an introducer valve coupled to the introducer sheath and having an inner tubular member, inserting a coupling projection of a treatment assembly into the inner tubular member of the introducer valve for less than a full length of the inner tubular member, the treatment assembly including a treatment chamber and a valve, and operating the introducer valve to seal the coupling projection of the treatment assembly to the inner tubular member.

According to another embodiment (“Example 28”), further to Example 27, the sheath of the introducer assembly is delivered into the body lumen of the patient prior to sealing the coupling projection to the inner tubular member.

According to another embodiment (“Example 29”), further to any of Examples 27-28, the method further includes operating the introducer valve between a sealed state and an unsealed state while the coupling projection is sealed to the inner tubular member.

According to another embodiment (“Example 30”), further to any of Examples 27-29, the method further includes introducing an endoluminal tool through the valve and treatment chamber of the treatment assembly and through the introducer assembly such that the introducer valve forms a hemostatic seal with the endoluminal tool and a valve of the treatment assembly forms a hemostatic seal with the endoluminal tool.

According to another embodiment (“Example 31”), further to any of Examples 27-30, the method further includes closing the valve of the treatment assembly, opening the introducer valve of the introducer assembly, withdrawing a bodily mass through the introducer valve and into the treatment chamber of the treatment assembly using the endoluminal tool while the treatment assembly is sealed to the introducer assembly, and closing the introducer valve of the introducer assembly such that the bodily mass is isolated from a bodily environment of the patient.

According to another embodiment (“Example 32”), further to Example 31, the method further includes directing a fluid through a recirculation loop coupled to the treatment chamber such that the fluid enters the treatment chamber and exits the treatment chamber through the recirculation loop.

According to another embodiment (“Example 33”), further to Example 32, the method further includes reintroducing a filtered portion of the fluid from the recirculation loop back into the body lumen of the patient.

According to one embodiment (“Example 34”), a method of treatment includes delivering an introducer assembly into a body lumen of a patient, the introducer assembly including an introducer sheath extending into the body lumen, coupling a treatment assembly to the introducer assembly, the treatment assembly including a first valve on a first end of the treatment assembly, a second valve on a second end of the treatment assembly and a treatment chamber extending therebetween, removing a bodily mass from body lumen of the patient, isolating the bodily mass from a bodily environment of the patient within the treatment chamber, and removing the treatment assembly from the introducer assembly with the bodily mass contained therein.

According to one embodiment (“Example 35”), a method of treatment includes delivering an introducer assembly into a body lumen of a patient, the introducer assembly including an introducer sheath extending into the body lumen and an introducer valve, coupling a first end of a treatment assembly to the introducer valve, the treatment assembly including a valve on a second end of the treatment assembly and a treatment chamber extending between the first end and the second end, unsealing the valve of the treatment assembly, delivering a device into the treatment chamber through the valve of the treatment assembly, sealing the valve of the treatment assembly, unsealing the introducer valve, advancing the device through the introducer valve for delivery to the body lumen.

According to another embodiment (“Example 36”), further to Example 35, the method further includes delivering a fluid to the treatment chamber to substantially remove entrapped air from the device.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows a side view of a treatment system, in accordance with an embodiment;

FIG. 2 shows a side view of a treatment assembly of the treatment system of FIG. 1, in accordance with an embodiment;

FIG. 3A shows an exploded view of an introducer valve of the treatment system of FIG. 2, in accordance with an embodiment;

FIG. 3B shows a cross-sectional view of the introducer valve of the treatment system, in accordance with an embodiment;

FIG. 3C shows another cross-sectional view of the introducer valve of the treatment system of FIG. 2, in accordance with an embodiment;

FIG. 4 shows a side-view of the introducer valve of the treatment system of FIG. 2, in accordance with an embodiment;

FIG. 5 shows a side view of a treatment assembly of the treatment system of FIG. 2, in accordance with an embodiment;

FIG. 6 shows another side view of the treatment assembly of the treatment system of FIG. 1 including a reservoir, in accordance with an embodiment;

FIG. 7 shows an alternative embodiment of the treatment assembly of the treatment system of FIG. 1, in accordance with an embodiment;

FIG. 8 shows a recirculation loop of the treatment assembly of FIG. 7, in accordance with an embodiment;

FIG. 9 shows a fluid flow diagram of the treatment system of FIG. 7, in accordance with an embodiment;

FIG. 10A shows a cross-sectional view of the introducer valve in an unsealed state prior to engagement with the treatment assembly of FIG. 2 or 7, in accordance with an embodiment;

FIG. 10B shows a cross-sectional view of the introducer valve in a sealed state prior to engagement with the treatment assembly of FIG. 2 or 7, in accordance with an embodiment;

FIG. 10C shows a cross-sectional view of the introducer valve in an unsealed state after engagement with the treatment assembly of FIG. 2 or 7, in accordance with an embodiment;

FIG. 10D shows a cross-sectional view of the introducer valve in a sealed state after engagement with the treatment assembly of FIG. 2 or 7, in accordance with an embodiment;

FIG. 11 illustrates an introducer assembly of the system of FIG. 1, inserted within a body lumen of the patient, in accordance with an embodiment;

FIG. 12 illustrates a valve of the treatment assembly of FIG. 1 being sealed, in accordance with an embodiment;

FIG. 13 illustrates an engagement of the introducer assembly and the treatment assembly of FIG. 1, in accordance with an embodiment;

FIG. 14 illustrates the treatment system of FIG. 1 being flushed with a fluid, in accordance with an embodiment;

FIG. 15 illustrates the treatment system of FIG. 1 being pressurized with the fluid, in accordance with an embodiment;

FIG. 16 illustrates an endoluminal tool within the treatment system of FIG. 1, in accordance with an embodiment;

FIG. 17 illustrates a bodily mass translated through the introducer valve of FIG. 1, in accordance with an embodiment;

FIG. 18 illustrates the bodily mass sealed within the treatment system, of FIG. 1 in accordance with an embodiment;

FIG. 19 illustrates the bodily mass within a visualization portion of the treatment system of FIG. 1, in accordance with an embodiment;

FIG. 20 illustrates the bodily mass translated through a valve of the treatment assembly of FIG. 1, in accordance with an embodiment;

FIG. 21 illustrates the bodily mass removed from the treatment system of FIG. 1, in accordance with an embodiment;

FIG. 22 illustrates the bodily mass in a partially dissolved state within a visualization portion of the treatment system of FIG. 1;

FIG. 23 illustrates the bodily mass sealed within the treatment system, of FIG. 7 in accordance with an embodiment;

FIG. 24 illustrates the treatment assembly of the system of FIG. 1, prior to receiving a delivery system, in accordance with an embodiment;

FIG. 25 illustrates the treatment assembly of the system of FIG. 1, receiving the delivery system within the treatment chamber, in accordance with an embodiment;

FIG. 26 illustrates the treatment assembly of the system of FIG. 1, with the delivery system sealed within the treatment chamber, in accordance with an embodiment;

FIG. 27 illustrates the treatment assembly of the system of FIG. 1, with the delivery system advanced through the treatment chamber, in accordance with an embodiment; and

FIG. 28 illustrates the treatment assembly of the system of FIG. 1, with the delivery system advanced through the introducer valve, in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

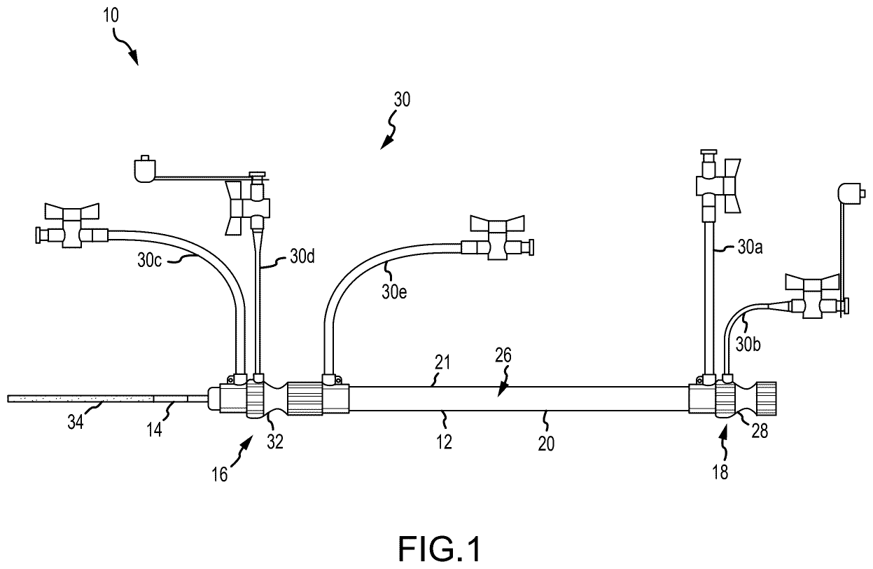

FIG. 1 shows a treatment system 10 including a treatment assembly 12 and an introducer assembly 14, according to some embodiments. As shown, the treatment system 10 includes a proximal valve assembly 16 positioned between the treatment assembly 12 and the introducer assembly 14 and a distal valve assembly 18 positioned on an end of the treatment assembly 12. The treatment assembly 12 may be both operable to be in fluid communication with, and removably coupled to, the introducer assembly 14.

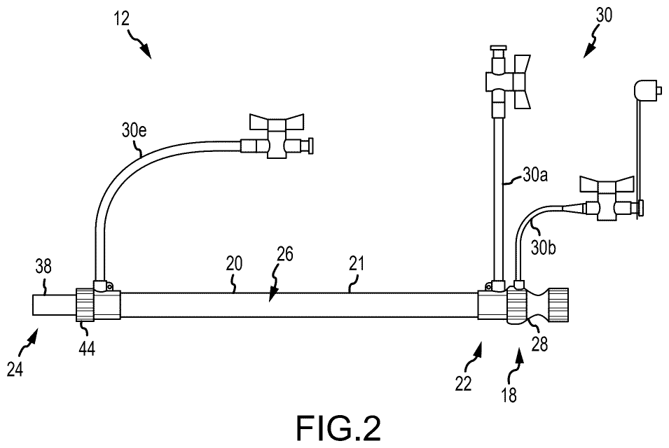

The treatment assembly 12 includes a treatment chamber 20 having a first end 22 (FIG. 2), a second end 24 (FIG. 2), and defining a chamber lumen 26 therebetween. The first end 22 of the treatment chamber 20 is in fluid communication with the distal valve assembly 18 when the distal valve assembly 18 is in an unsealed state such that the chamber lumen 26 is in fluid communication with an inner lumen of the distal valve assembly 18. As shown, the distal valve assembly 18 includes a valve 28 coupled to the first end 22 of the treatment chamber 20. The valve 28 and the first end 22 of the treatment chamber 20 form the distal valve assembly 18. The valve 28 is transitionable between the unsealed state and a sealed state such that the valve 28 is operable to seal the chamber lumen 26 at the first end 22 of the treatment chamber 20. The treatment system 10 may include one or more ports 30 including, but not limited to, one or more flush ports and one or more valve ports. For example, one or more ports 30, including a flush port 30a, may be coupled to the treatment chamber 20 or portions of the valve 28 such that the flush port 30a is in fluid communication with the chamber lumen 26. A valve port 30b may be in fluid communication with the valve 28 and operable to receive a syringe 58 therein to transition the valve 28 between the sealed and unsealed states. In some embodiments, portions of the flush port 30a (e.g., a valve) may have different colors than portions of the valve port 30b (e.g., a valve) to allow a user to quickly distinguish between the different ports.

The second end 24 of the treatment chamber 20 is in fluid communication with the proximal valve assembly 16 when the proximal valve assembly 16 is in an unsealed state, such that the chamber lumen 26 is in fluid communication with the proximal valve assembly 16. The introducer assembly 14 includes an introducer valve 32 fluidly coupled to the second end 24 of the treatment chamber 20, where the introducer valve 32 and the second end 24 of the treatment chamber 20 form the proximal valve assembly 16. Similar to the valve 28, the introducer valve 32 may be coupled to and in fluid communication with a flush port 30c and a valve port 30d.

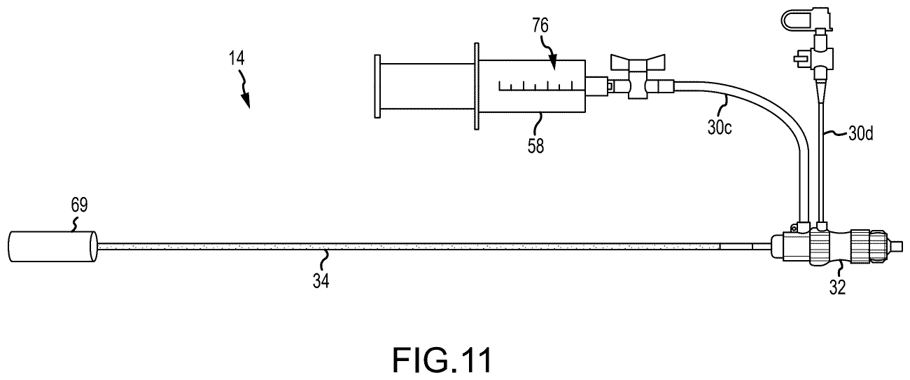

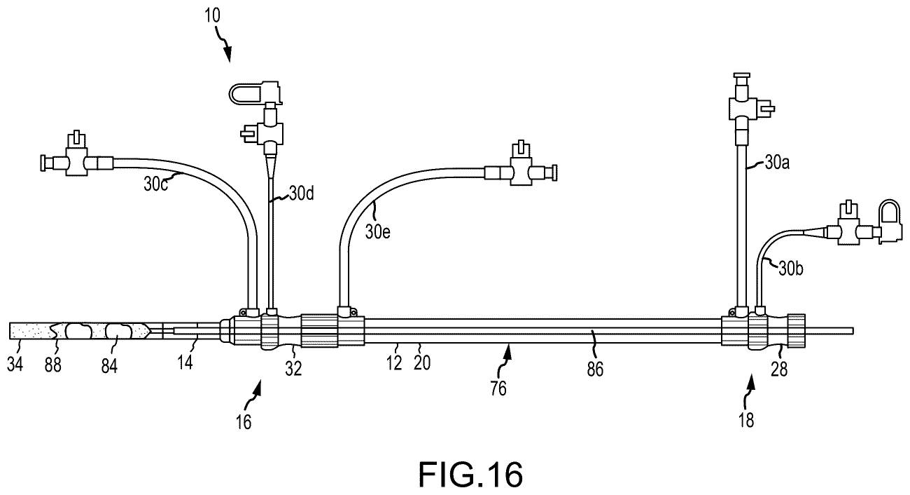

The introducer assembly 14 may further include an introducer sheath 34 in fluid communication with the introducer valve 32. The introducer sheath 34 may be operable to extend within a body lumen 69 of a patient (FIG. 11) and may extend distally from the introducer valve 32. The introducer sheath 34 defines a length and a sheath lumen extending along the length. When the introducer valve 32 is in the unsealed state, the introducer sheath 34 and the sheath lumen may be in fluid communication with the introducer valve 32 and the chamber lumen 26. The introducer sheath 34 may be configured to be insertable into the body lumen 69 of a patient. In some embodiments, an endoluminal device 36 may be delivered to the body lumen 69 via the treatment system 10. The endoluminal device 36 may extend through the introducer sheath 34 and one or both of the proximal valve assembly 16 and the distal valve assembly 18. In some embodiments, and as further described below, a bodily mass 84 (FIG. 16) may be removed from the body lumen 69 via the treatment system 10.

FIG. 2 shows the treatment assembly 12 of the treatment system 10. At least a portion of the treatment chamber 20 includes a visualization portion 21 which may be one of clear, translucent, or transparent material to permit a user to observe interior surfaces of the chamber lumen 26 though the treatment chamber 20 during the surgical procedure. As further discussed below, visualization of an internal surface of the chamber lumen 26 can assist a user during surgical procedures. Optionally, the one or more ports 30 of the treatment system 10 may be positioned within the visualization portion 21 such that a user can observe fluid flow into and out of the treatment chamber 20. As further shown in FIG. 2, the second end 24 of the treatment chamber 20 includes a coupling projection 38 configured to be received within a portion of the introducer valve 32.

In some embodiments, a length of the treatment chamber 20 is between approximately 10 cm to 20 cm, optionally approximately 10 cm, optionally between approximately 10 cm and 12 cm, optionally between approximately 14 cm and 16 cm, optionally between approximately 16 cm and 18 cm, optionally between approximately 18 cm and 20 cm, or optionally approximately 20 cm, or optionally any range or value between any of the foregoing ranges and values.

In some embodiments, the treatment chamber 20 may be configured to longitudinally collapse and/or diametrically collapse when the treatment system 10 is pressurized. For example, the treatment chamber 20 may include a plurality of pleats including one or more longitudinal pleats extending along a longitudinal axis of the treatment chamber 20 configured to facilitate longitudinal collapse. Additionally, or alternatively, the treatment chamber 20 may include one or more diametric pleats extending along a circumferential axis of the treatment chamber 20 configured to facilitate diametric collapse.

The treatment chamber 20 may be formed of a polymeric material. The polymeric material may include polytetrafluoroethylene (PTFE), expanded polytetrafluoroethylene (ePTFE), fluorinated ethylene propylene (FEP), polyethylene (e.g., one or more of low-density polyethylene (LDPE), high-density polyethylene (HDPE), ultra-high molecular weight polyethylene (UHMWPE) and/or expanded polyethylene), polycarbonate; ethylene-vinyl acetate (EVA); polyurethane; poly(tetramethyl-p-silphenylene siloxane) polysiloxane (PTMPS), optically transparent polyimide (CPI), and/or any other suitable material. In some embodiments, the treatment chamber 20 may be configured to collapse upon application of an external force (e.g., manual pinching by a user) or internal force (e.g., vacuum pressure).

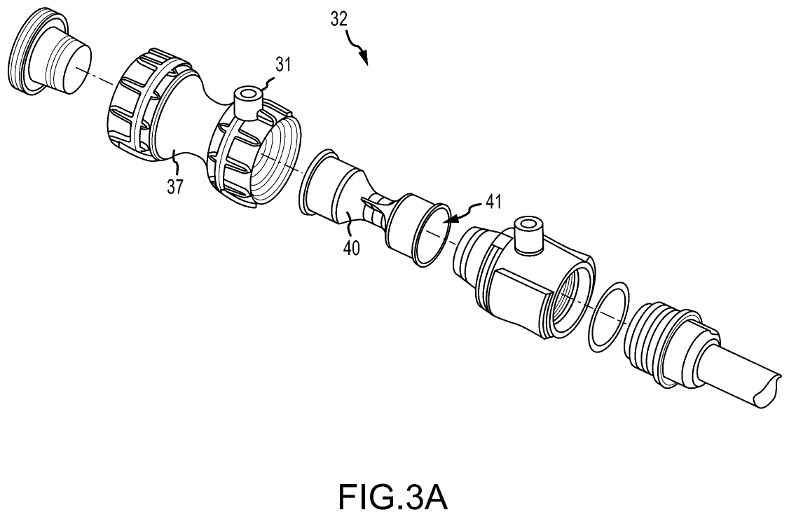

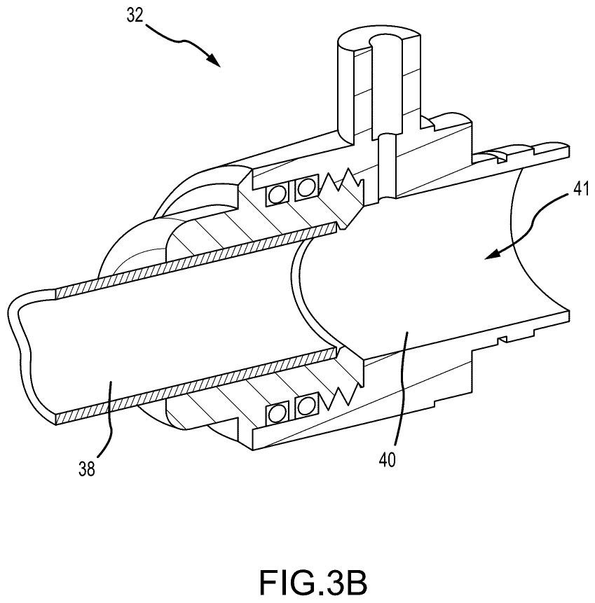

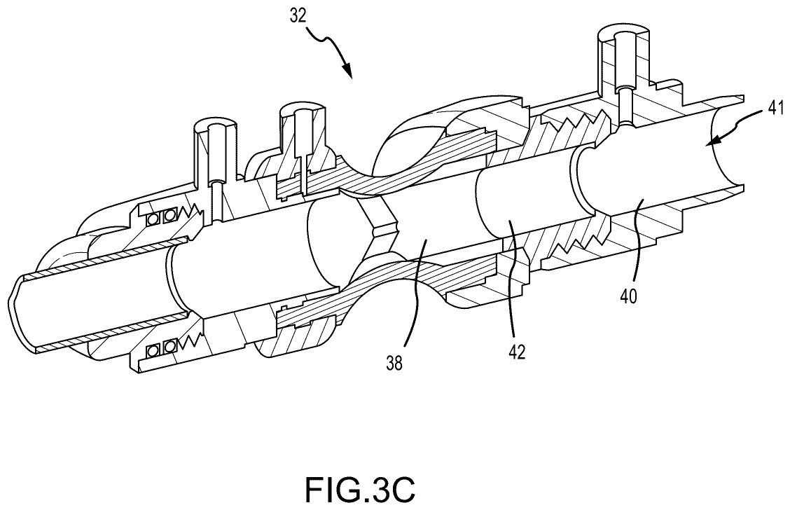

FIG. 3A shows an exploded view of the introducer valve 32. FIGS. 3B-3C show an engagement of the treatment assembly 12 and the introducer assembly 14.

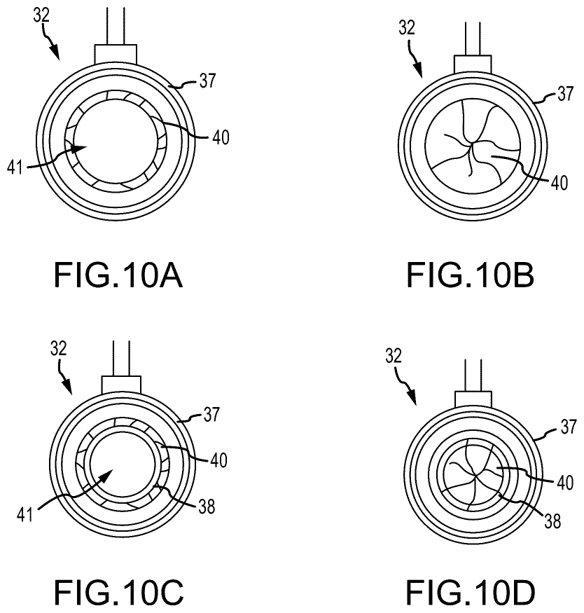

As shown in FIG. 3A, the introducer valve 32 includes an outer tubular member 37 and an inner tubular member 40. The outer tubular member 37 may include a connection 31 to valve port 30d. The inner tubular member 40 may be connected to the outer tubular member 37 by methods including, but not limited to, insertion molding, interference fit, adhesion or adhesives, ultrasonic welding and thermal bonding. The outer tubular member 37 may be constructed of any elastomer, latex or polycarbonate with desirable mechanical and biocompatible properties. In one embodiment, outer tubular member 37 comprises silicone and has an hourglass shape when not in the pressurized, or sealed state. In some examples, when pressurized, the hourglass shape of outer tubular member 37 becomes distended to indicate pressurization (e.g., a desirable pressure) in a region or space between the outer tubular member 37 and the inner tubular member 40. The region between the outer tubular member 37 and the inner tubular member 40 (also described as a pressurizable space) can be pressurized by injecting saline into the region using a syringe 58, which can be facilitated by the valve port 30d. When the region is pressurized, the inner tubular member 40 may collapse (FIGS. 10A-10D) to seal the introducer valve 32 and transition the introducer valve 32 to a sealed state and prevent back bleeding through the introducer valve 32.

The inner tubular member 40 may be constructed of any thin, strong, compliant (e.g., drape-able) material such as ePTFE, fabrics, silk, or Kevlar® brand fiber. These materials may be used as a single layer construct or a multi-layer construct. In one embodiment, inner tubular member 40 can be formed of a thin porous substrate, including but not limited to polymeric materials such as polytetrafluoroethylene (PTFE) or expanded polytetrafluoroethylene (ePTFE). This construct may be comprised of multiple layers of the thin, porous substrate, the pores of which may be filled or imbibed with a polymer. The filling or imbibing polymer may be the same as the construct or may be a different polymer.

As shown in FIGS. 3B-3C, the inner tubular member 40 is configured to receive the coupling projection 38 therein such that the coupling projection 38 is sealed to the inner tubular member 40 and the inner tubular member 40 remains operable to be transitioned between the sealed state and the unsealed state. In other words, the coupling projection 38 does not interfere with sealing the introducer valve 32 such that the introducer valve 32 is still operable to seal and unseal an inner lumen of the introducer valve 32 when the coupling projection 38 is engaged therein. As such, when the introducer valve 32 is in the unsealed state, the introducer valve 32 is operable to seal the chamber lumen 26 at the second end 24 of the treatment chamber 20 of when the coupling projection 38 is received within the inner tubular member 40.

The inner tubular member 40 may be constructed of any thin, strong, compliant (e.g., drapeable) material such as ePTFE, expanded polyethylene, fabrics, silk, or Kevlar® brand fiber, for example. Such materials may be used as a single layer construct or a multi-layer construct as appropriate.

As shown in FIG. 3C, the treatment assembly 12 may further include a gasket 42 (e.g., an O-ring) associated with the coupling projection 38. The gasket 42 may be receivable within the inner tubular member 40 and operable to seal to the inner tubular member 40 with the coupling projection 38 inserted within the inner tubular member 40. The gasket 42 may be positionable about the coupling projection 38 such that the gasket 42 is between the inner tubular member 40 and the coupling projection 38. In some embodiments, the gasket 42 is inflatable to seal between the inner tubular member 40 and the coupling projection 38.

In some embodiments, the coupling projection 38 comprises a relatively tacky and/or textured material. The tacky and/or textured material can facilitate sealing between the coupling projection 38 and the inner tubular member 40. In other embodiments, the coupling projection 38 comprises a relatively smooth material. The coupling projection 38 may also be relatively rigid to help facilitate effective sealing.

In some embodiments, the coupling projection 38 may have a diameter between approximately 5 French and 26 French, optionally approximately 5 French, between approximately 5 French and 10 French, between approximately 10 French and 15 French, between approximately 15 French and 20 French, between approximately 20 French and 26 French, or approximately 26 French.

In some embodiments, only a portion of a length of the coupling projection 38 may be inserted within the inner tubular member 40. For example, the inner tubular member 40 may have a shorter length relative to the length of the coupling projection 38 such that less than a full length of the coupling projection 38 can be received therein. For example, optionally 75% or less of the full length, optionally 50% or less of the full length, optionally 25% or less of the full length, optionally 10% or less of the full length of the coupling projection 38 is inserted within the inner tubular member 40. The full length of the coupling projection 38 may between approximately 1 cm to 5 cm in length, optionally approximately 1 cm, between approximately 1 cm and 2 cm, between approximately 2 cm and 3 cm, between approximately 3 cm and 4 cm, between approximately 4 cm and 5 cm, or optionally approximately 5 cm.

In other embodiments, the full length of the coupling projection 38 may approximately equal to, or shorter than, a full length of the inner tubular member 40 such that the full length of the coupling projection 38 may be received within the inner tubular member 40. In such embodiments, the length of the coupling projection 38 may be optionally 50% or less of the length of the inner tubular member 40, optionally 25% or less of the length of the inner tubular member 40, optionally 10% or less of the length of the inner tubular member 40.

In some embodiments, the coupling projection 38 includes one or more visual markings 39 demarcating a desired insertion position of the coupling projection 38 into the introducer valve 32. The visual markings 39 may be pre-designated by a manufacturer along at least a portion of the length of the coupling projection 38 and corresponding to one or more lengths of the inner tubular member 40. In other embodiments, the visual markings may be made by a user during a procedure when the treatment assembly 12 and introducer assembly 14 are being coupled together. In this way, the insertion length of the coupling projection 38 may be customized based, at least in part, on dimensions of the introducer assembly 14 used.

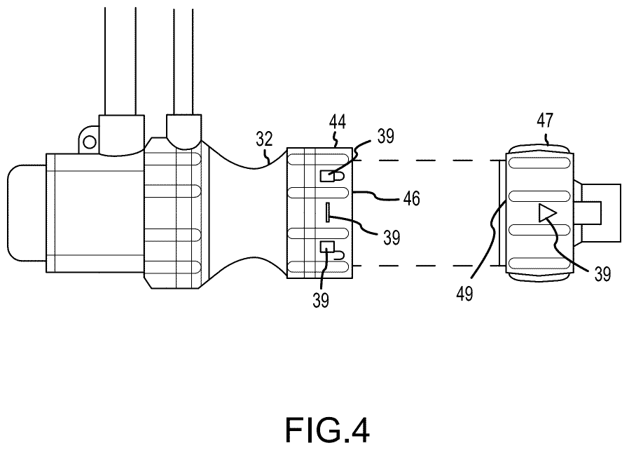

As shown in FIG. 2, the treatment assembly 12 may include a collar 44 associated with the coupling projection 38 and positioned between the coupling projection and the treatment chamber 20. The collar 44 is configured as a coupling mechanism operable to engage portions of the introducer assembly 14 and facilitate coupling between the treatment assembly 12 and the introducer assembly 14. The collar 44 may engage a complementary fixture 47 of the introducer assembly 14, as shown in FIG. 4. The collar 44 may include a mating feature 46 operable to mate with a complementary feature 49 of the introducer assembly 14 to longitudinally fix a position of the treatment assembly 12 relative to the introducer assembly 14. In some embodiments, the mating feature 46 includes one or more projections and the complementary feature 49 is one or more recesses. The collar 44 may couple to the complementary fixture 47 via mechanisms including, but not limited to, complementary threads, adhesives, snap fits, and/or fasteners to couple outer portions of the treatment assembly 12 and introducer assembly 14 together. The reverse configuration is also contemplated in which the collar 44 defines one or more recesses. A portion of the collar 44 may be rotatable (e.g., an outer portion) such that the mating feature 46 is rotatable to mate with the complementary feature of the introducer assembly 14. The collar 44 and/or the complementary fixture 47 may also include visual markings 39 such as symbols, including but not limited to arrows, locks, or unlocked locks, indicating an insertion end or an unlocked/locked configuration. In some embodiments, the visual markings 39 indicate to the user which rotation direction locks/unlocks the collar 44 to the complementary fixture 47. Although one or more projections may be used, other mating features (e.g., complementary threads) may also be employed for the collar 44 and/or the complementary fixture 47. And, although portions of the collar 44 may be rotatable, rotation of other portions of the treatment assembly 12 and/or the introducer assembly 14 is limited to maintain proper sealing between the components. The engagement facilitated by the collar 44 and the complementary fixture 47 may be completed after inserting the coupling projection 38 within the inner tubular member 40.

In some embodiments, and further to the above, the collar 44 is operable to engage the introducer valve 32 to control and/or limit an insertion distance of the coupling projection 38 that is inserted within the inner tubular member 40. The collar 44 may limit the insertion distance of the coupling projection 38 between approximately 1% and 100% of the length of the coupling projection 38. In some embodiments, only a portion of the full length is inserted within the inner tubular member 40, optionally 75% or less of the full length, optionally 50% or less of the full length, optionally 25% or less of the full length, optionally 10% or less of the full length, or optionally any value or range of values between any of the foregoing values or range of values. In some embodiments, the collar 44 may be positioned between approximately 2 cm and 5 cm from the second end 24 of the treatment chamber 20, where the position of the collar 44 is at least partially dependent on the insertion length of the coupling projection 38. The position of the collar 44 may be predetermined (e.g., at the manufacturer) or may be adjusted by the user when coupling the treatment assembly 12 and introducer assembly 14. The adjustment of the position of collar 44 along the treatment chamber 20 can facilitate use of the treatment assembly 12 with different geometries and dimensions of the introducer assembly, as discussed above.

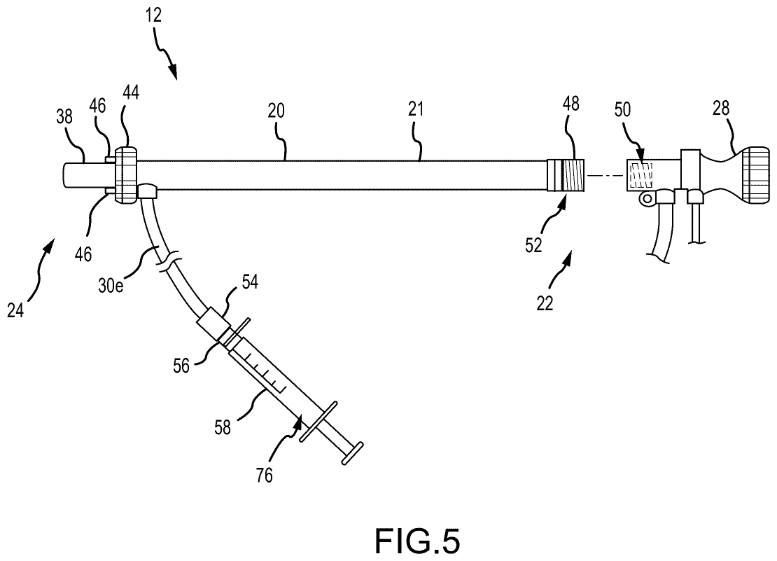

FIG. 5 shows the treatment assembly 12 of the treatment system 10 disengaged from the valve 28. As shown, the first end 22 of the treatment chamber 20 defines a projection 48 expending proximally from a remainder of the treatment chamber 20 and operable to couple to a complementary portion 50 of the valve 28.

In some embodiments, the projection 48 includes threads operable to be received by complementary threads of the complementary portion 50. In some embodiments, an adhesive material is applied to the projection 48 to facilitate coupling between the treatment assembly 12 and the valve 28. The projection 48 may define a recessed portion 52 that extends diametrically around the projection 48 that is operable to receive a gasket (e.g., an O-ring) therein. When the treatment chamber 20 is coupled to the valve 28, the gasket is positioned between the treatment assembly 12 and the valve 28 to facilitate a fluid-tight seal between the components.

As shown in FIG. 5, the one or more of the ports 30, including the flush port 30a, may include a two-way valve 54 and a fitting 56. The fitting 56 may be a Luer slip fitting or a Luer lock fitting and operable to receive a syringe 58 thereon. The two-way valve 54 can facilitate moving fluid (e.g., saline) into the treatment chamber 20 in a first configuration and can facilitate moving fluid out from the treatment chamber 20 in a second configuration. As discussed above, the flush port 30e may be configured to deliver fluid to the chamber lumen 26 within the visualization portion 21 of the treatment chamber 20. Although one flush port 30e is shown in FIG. 5, the other flush ports of the one or more ports 30 may have a similar configuration.

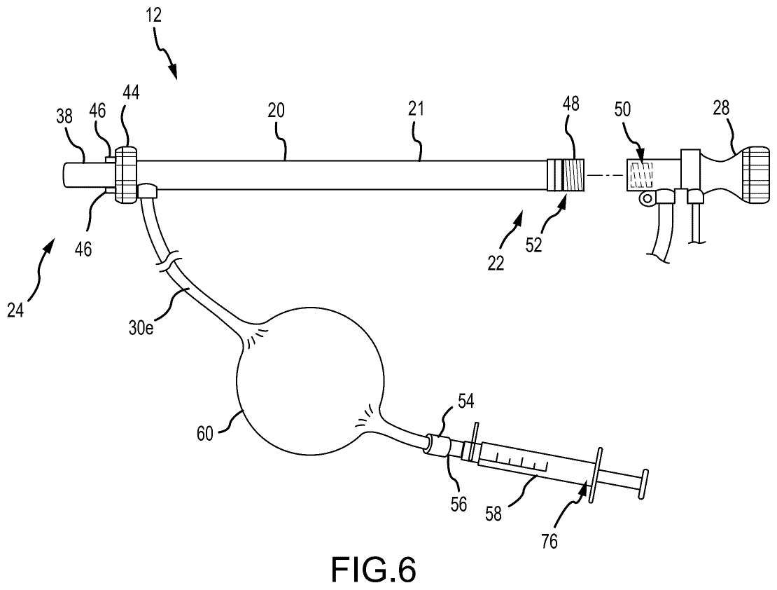

Turning to FIG. 6, in some embodiments, the one or more ports 30 may include a reservoir 60 operable to move fluid from the reservoir 60 to the chamber lumen 26. The reservoir 60 may be positioned along a length of the flush port 30e and configured to receive fluid therein from the syringe 58. The reservoir 60 may be refillable and configured to expand when filled with fluid and collapsible when emptied of fluid. When the reservoir 60 is collapsed, the user may quickly recognize that additional fluid is needed to refill and re-expand the reservoir 60. The two-way valve 54 can contain the fluid within the reservoir 60 in the absence of the syringe 58. Fluid may be expelled from the reservoir 60 and to the treatment chamber 20 upon application of pressure (e.g., applying hand pressure or squeezing force) or creation of a negative pressure within the treatment assembly 12. Although the reservoir 60 is shown with respect to flush port 30e, the reservoir 60 may be used with any of the fluid ports, including those coupled to the treatment system 10 at the valve 28 and/or the introducer valve 32.

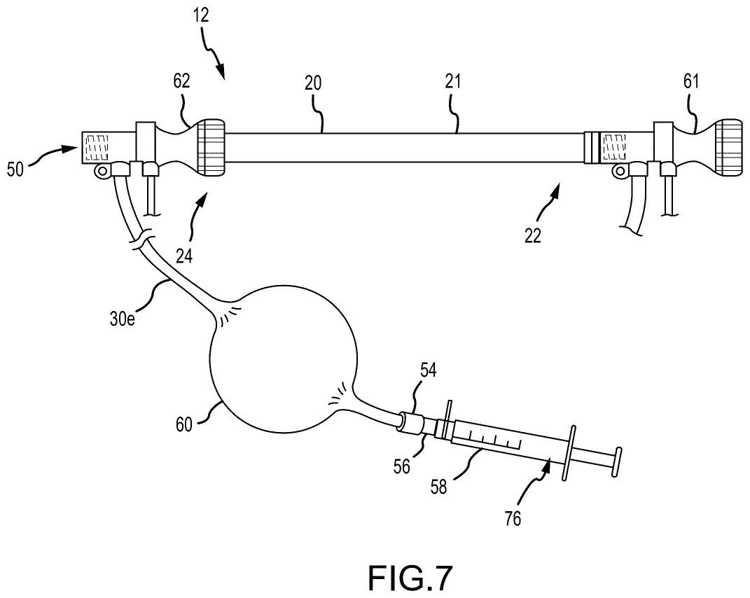

Turning to FIG. 7, in alternative embodiments of the treatment system 10, in place of the introducer assembly 14, a second valve 62 may be coupled to the second end 24 of the treatment chamber 20. Although described as an alternative, features of the FIG. 7 embodiment may be interchanged with features of the treatment systems 10 that include the introducer assembly 14 (e.g., as shown in FIG. 2), and vice versa.

As shown in FIG. 7, the second valve 62 may be substantially similar to the valve 28. Similar to the introducer valve 32, the coupling projection 38 may be inserted into the second valve 62 such that the second valve 62 is sealable to the coupling projection 38 while being operable to seal and unseal an inner lumen of the second valve 62. The valve 28 and the second valve 62 are operable to seal and unseal independently from each other.

As shown, the treatment chamber 20 is sandwiched between a first valve 61 and a second valve 62. The valve 28 of FIG. 2, the first valve 61, and the second valve 62 may each be substantially similar to each other. In some embodiments, the second valve 62 defines the projection 48 that is operable to couple to a second treatment assembly (not shown), similar to the treatment assembly 12. In this manner, two or more treatment assemblies 12 may be coupled together in series and may be used to receive portions of the endoluminal device 36 and/or a bodily mass 84 (FIG. 16) therein. The first valve 61 and the second valve 62 are operable to seal and unseal independently of each other. As subsequently described, this facilitates movement of material from the first end of the treatment chamber to the second end of the treatment chamber and vice versa.

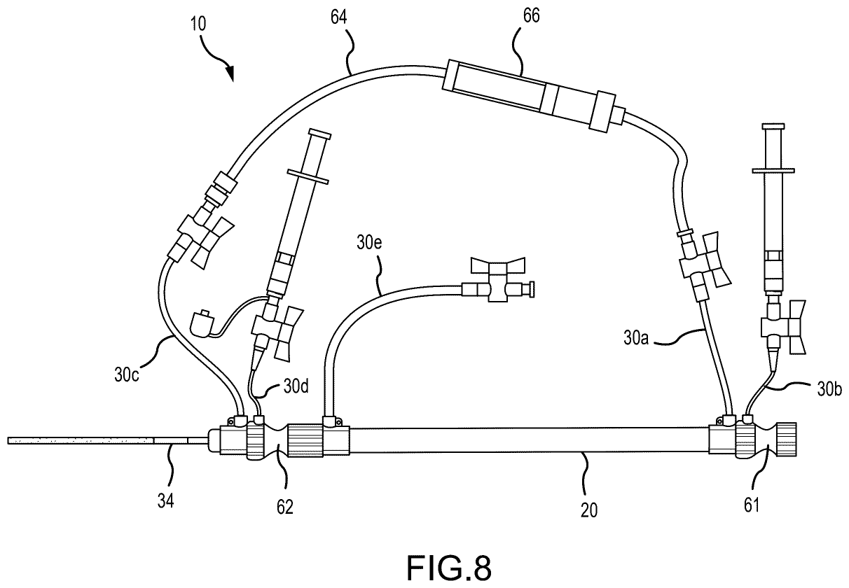

In some embodiments, as shown in FIG. 8, the treatment system 10 includes a recirculation loop 64. Although shown with respect to the embodiment of FIG. 7, the recirculation loop 64 can be implemented with any of the treatment systems 10 disclosed herein, including treatment systems 10 having the introducer assembly 14 (e.g., as shown in FIG. 2). As shown, the recirculation loop 64 is in fluid communication with the treatment chamber 20 and the chamber lumen 26 such that fluid passing into the treatment chamber 20 is directed out of the treatment chamber 20 through the recirculation loop 64. The fluid may include bodily fluids 78, such as blood, that enters the treatment chamber 20 during a surgical procedure. The bodily fluid 78 may be directed through the treatment chamber 20 and into the recirculation loop 64 for introduction back to the patient. As shown, the recirculation loop 64 may include at least one filter 66 to filter the bodily fluid 78 prior to reintroduction to the patient (or treatment chamber 20 as applicable). In some embodiments, the bodily fluid 78 is reintroduced through the introducer valve 32 during or after the surgical procedure. Inclusion of the recirculation loop 64, and delivery of the bodily fluid 78 back to the patient during or after the surgical procedure, decreases overall blood loss for the patient.

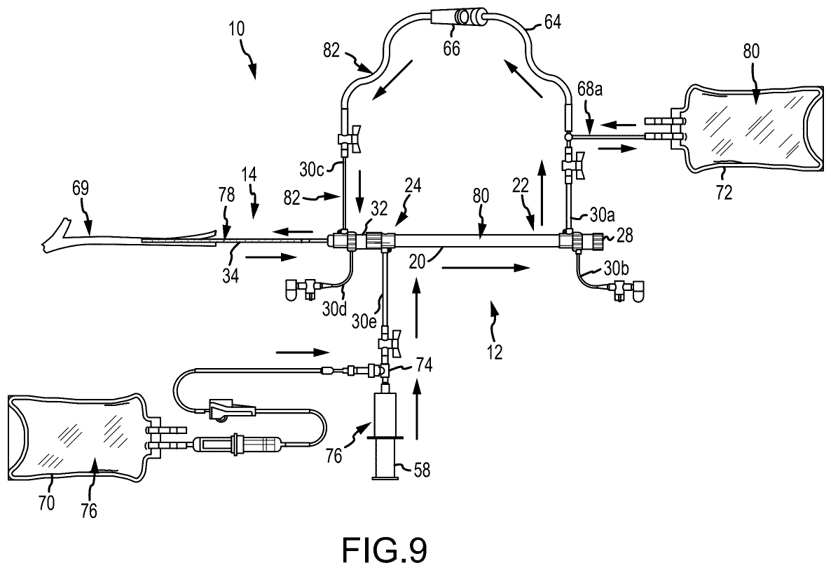

FIG. 9 illustrates movement of fluid through the treatment system 10, including through the recirculation loop 64. Although shown with respect to the embodiment of FIG. 7, the recirculation loop 64 can be implemented with any of the treatment systems 10 disclosed herein, including treatment systems 10 having the introducer assembly 14 (e.g., as shown in FIG. 2). As shown, a second end 24 of the treatment chamber 20 is in fluid communication with the flush port 30e. The flush port 30e is in fluid communication with one or both of a syringe 58 and a fluid bag 70. The syringe 58 and fluid bag 70 may both include the same fluid 76, such as saline, or may include different fluids. Fluid 76 from one or both of the syringe 58 and the fluid bag 70 is delivered to the treatment chamber 20. For example, when both the valve 28 and introducer valve 32 are in the sealed state, and the remaining one or more ports 30 are closed, the introduction of the fluid 76 can pressurize the treatment system 10 and remove air therefrom. During the surgical procedure, and as further discussed below, the introducer valve 32 may be unsealed to allow the bodily mass 84 (FIG. 16) to enter the treatment chamber 20 through the introducer valve 32. When receiving the body mass, bodily fluid 78 (e.g., blood) from the patient will also enter the treatment chamber 20. The bodily fluid 78 and fluid 76 from one or both of the syringe 58 and the fluid bag 70 may mix together within the treatment chamber 20 to form a fluid mixture 80 and the fluid mixture 80 may be directed from the first end 22 of the treatment chamber 20 to the recirculation loop 64. The fluid mixture 80 may primarily comprise the bodily fluid 78.

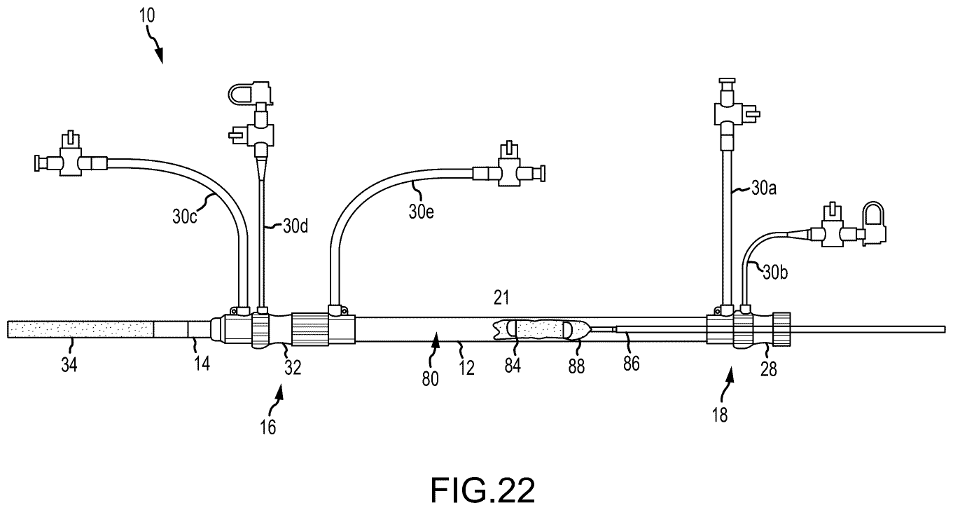

The inclusion of fluid 76, such as saline, within the fluid mixture 80 and within the treatment system 10 may have multiple functions. For example, blood is opaque and saline is relatively clear, so introduction of saline helps to enable visualization of the contents within the treatment system 10 for viewing by the user. Movement of saline through the treatment chamber 20 may also help longitudinally displace the blood through the treatment chamber 20 and direct blood toward the recirculation loop 64 and filter 66 for reintroduction to the patient's vasculature. The saline and may also reduce viscosity of the fluid mixture 80 for ease of flow through the treatment system 10. Additionally, the saline may assist with treatment and/or separation (e.g., dissolution) of a bodily mass 84. For example, and as shown in FIG. 22, saline alone, or saline supplemented with an active ingredient (e.g., an enzyme), may help treat or dissolve portions of the bodily mass 84 within the treatment chamber 20.

The recirculation loop 64 may include one or more ports 68, including flush ports 68a, where the one or more ports 68 are operable to couple to a collection bag 72. The collection bag 72 may be in fluid communication with the recirculation loop 64 with a three-way valve 74, such as a check flow switch. The collection bag 72 may store the fluid mixture 80 during the surgical procedure until the user is ready to reintroduce the fluid mixture 80 back to the patient. When ready for reintroduction, the three-way valve 74 may allow the fluid mixture 80 to continue along the recirculation loop 64 to the filter 66. The filter 66 is operable to remove particulates from the fluid mixture 80 and store the particulates within the filter 66 to form a filtered fluid 82 (referred to herein as a filtered portion of the fluid). The filtered fluid 82 is then directed back to the introducer valve 32 and is directed back to the patient through the introducer assembly 14.

FIGS. 10A-10D illustrate a cross-sectional view of the introducer valve 32 in the unsealed and sealed states. In particular, FIGS. 10A-10D illustrate an end view of the introducer valve 32 at the second end of the treatment chamber 20 and at the engagement of the inner tubular member 40 and the coupling projection 38, with other portions of the treatment chamber 20 and treatment system 10 removed for illustration purposes. FIG. 10A illustrates the unsealed state prior to the coupling projection 38 being received within the inner tubular member 40. In the unsealed state, the introducer valve 32 defines an inner lumen 41 therethrough. FIG. 10B illustrates the sealed state prior to the coupling projection 38 being received within the inner tubular member 40. As shown, the inner tubular member 40 may be configured to collapse in diameter when the introducer valve 32 is in the sealed state (e.g., when the treatment system 10 is pressurized). When collapsed, the inner tubular member 40 seals the inner lumen 41 to prevent fluid flow therethrough. FIG. 10C illustrates the unsealed state with the coupling projection 38 received within the inner tubular member 40. As discussed above, the coupling projection 38 does not interfere with the ability of the introducer valve to transition between the sealed and unsealed states. In particular, and as shown in FIG. 10C, the inner lumen 41 remains available in the unsealed state when the coupling projection 38 is received therein. FIG. 10D illustrates the sealed state with the coupling projection 38 received within the inner tubular member 40. In embodiments where the inner tubular member 40 is collapsible, the coupling projection 38 may also be configured to be collapsible (e.g., diametrically collapsible) to maintain the engagement with the inner tubular member 40. The valve 28 may include unsealed and sealed states similar to those shown in FIGS. 10A-10B, respectively.

Turning to FIGS. 11-22, methods of preparing the treatment system 10 for surgical procedures are described. In one embodiment, the treatment system 10 is used in a thrombectomy procedure, in which the bodily mass 84 (FIG. 16), such as a thrombus, is retrieved and removed from the body lumen 69 of the patient (e.g., a vessel). In other embodiments, a previously implanted endoluminal device, or at least a portion of the previously implanted endoluminal device, may be removed from the body lumen 69 of the patient following a similar procedure.

FIG. 11 shows the introducer assembly 14 delivered into the body lumen 69 of the patient. In particular, the introducer sheath 34 of the introducer assembly 14 extends within the body lumen 69. The introducer sheath 34 may be delivered to the body lumen 69 prior to coupling the treatment assembly 12 to the introducer valve 32, or in other words, prior to sealing the coupling projection 38 to the inner tubular member 40 of the introducer valve 32. A dilator (not shown) may be used to assist in delivering the introducer sheath 34 to the body lumen 69. The dilator may be delivered through the introducer valve 32 and through a lumen of the introducer sheath 34 when the introducer valve 32 is in the sealed state (e.g., as shown in FIG. 10B). Once the introducer sheath 34 is in position, the dilator may be removed from the introducer assembly 14 through the introducer valve 32 when the introducer valve 32 is in the sealed state. Once the dilator is removed, the introducer sheath 34 may be flushed and pressurized. In some embodiments, the introducer sheath 34 is flushed by delivery of fluid 76 through the flush port 30a coupled to the introducer assembly 14. The fluid 76 (e.g., saline) may be provided to the flush port 30a via the syringe 58.

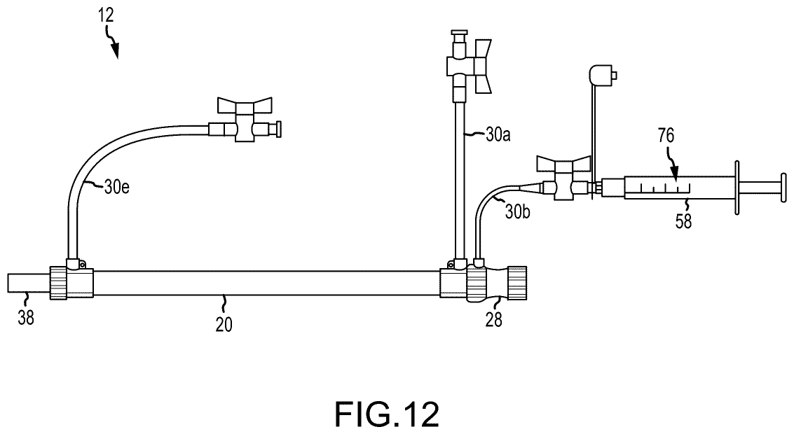

FIG. 12 shows the treatment assembly 12 of the treatment system 10 prior to coupling to the introducer assembly 14. Prior to coupling to the introducer assembly 14, the valve 28 of the treatment assembly 12 is sealed (e.g., as shown in FIG. 10B). In some embodiments, the valve 28 may be inflated similarly to introducer valve 32 as described with respect to FIG. 3A and FIGS. 10A-10B, and the valve 28 may sealed by inflating internal portions of the valve 28 with fluid 76. The valve 28 may be in fluid communication with a valve port 30b of the one or more ports 30, and the fluid 76 may be delivered to the valve 28 through the valve port 30b. The fluid 76 (e.g., saline) may be provided to the valve port 30b via the syringe 58.

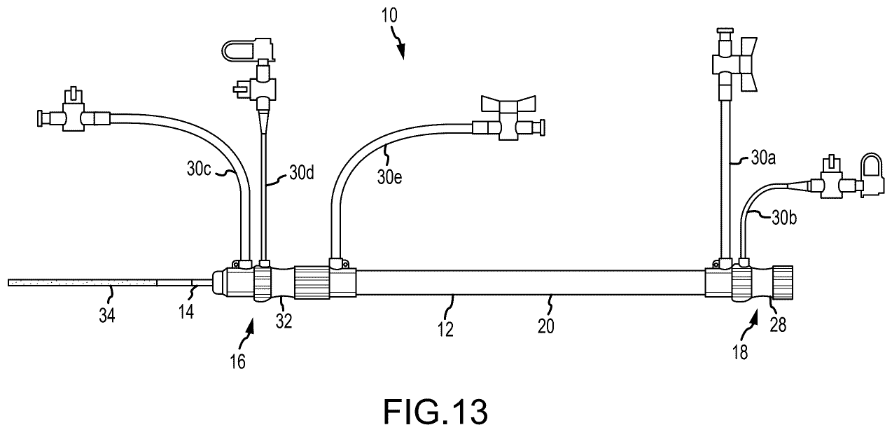

FIG. 13 shows the treatment assembly 12 coupled to the introducer assembly 14. To couple the two assemblies, and as discussed with respect to FIGS. 2-3C, the user operates the introducer valve 32 to seal the coupling projection 38 to the inner tubular member 40, which may include inserting the coupling projection 38 into the inner tubular member 40 of the introducer valve 32. The coupling projection 38 may be inserted within the inner tubular member 40 at less than a full length of the coupling projection 38. To receive the coupling projection 38, the introducer valve 32 is transitioned to an unsealed state (e.g., as shown in FIG. 10A) and the coupling projection 38 is inserted therein. The introducer valve 32 may be sealed once the coupling projection 38 is inserted therein (e.g., as shown in FIG. 10C). The introducer valve 32 remains operable to transition between the seal and unsealed states while the coupling projection 38 is coupled to the inner tubular member 40. In some embodiments, the collar 44 may be used to lock the longitudinal position of the treatment assembly 12 and the introducer assembly 14 once the coupling projection 38 is received within the introducer assembly 14. In some embodiments, the gasket 42 (FIG. 3B) may be receivable within the inner tubular member 40 when the coupling projection 38 is inserted therein. The gasket 42 may be operable to inflate to seal between the inner tubular member 40 and the coupling projection 38.

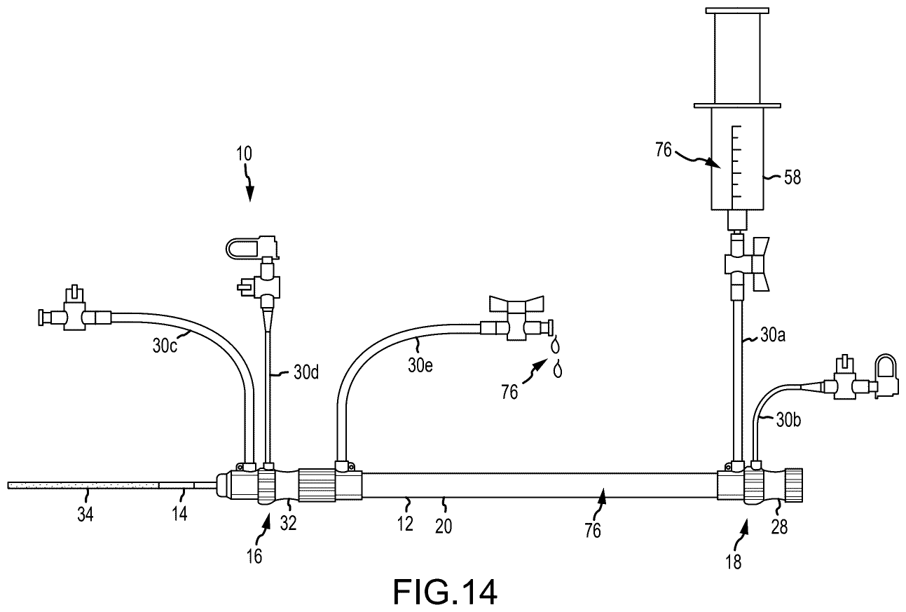

FIG. 14 shows the treatment assembly 12 being flushed with the fluid 76. While both the valve 28 and introducer valve 32 are in the sealed state (e.g., as shown in FIGS. 10B and 10D, respectively), the treatment assembly 12 may be flushed with fluid 76. The syringe 58 may be coupled to the flush port 30a, and a flush port 30e may be open, such that the user delivers fluid 76 from the syringe 58 through the treatment chamber 20, and out through the flush port 30e. While flushing, the introducer valve 32 is sealed (e.g., as shown in FIG. 10D) such that the fluid 76 does not travel therethrough. In some embodiments, flushing with the fluid 76 helps to flush air from the treatment system 10.

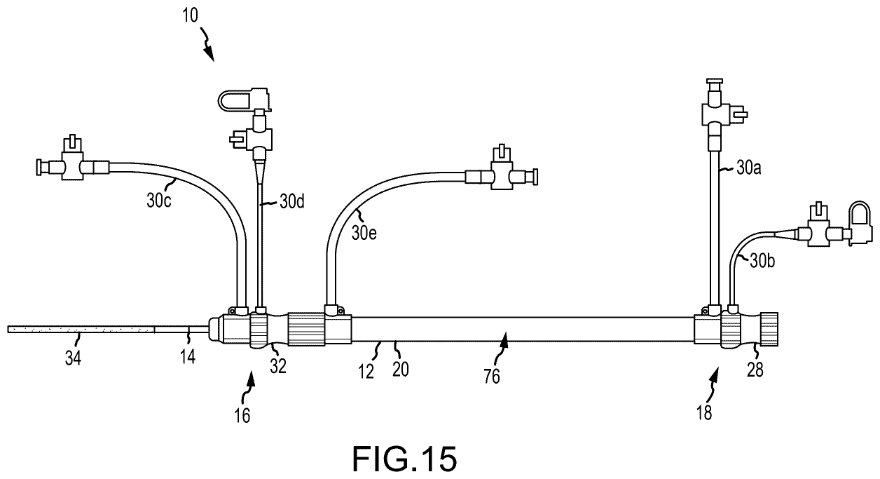

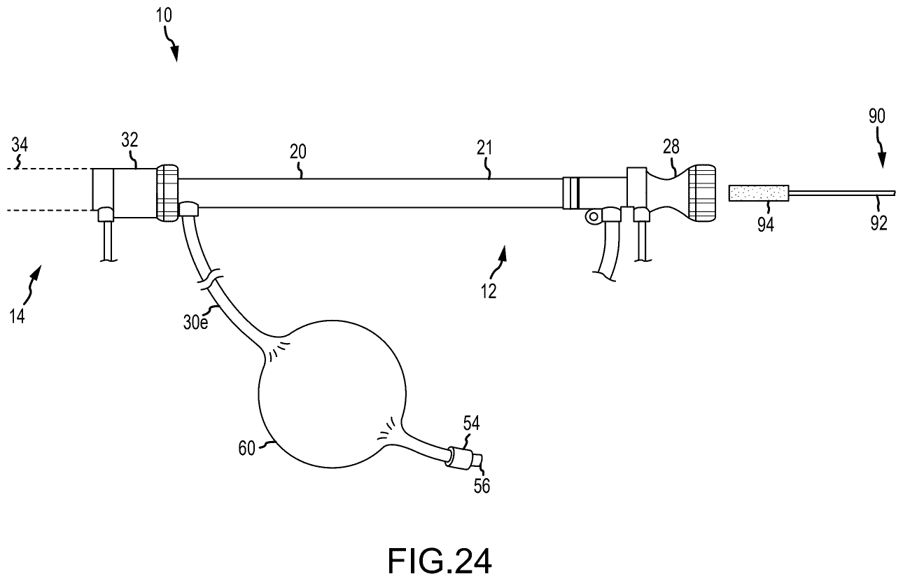

FIG. 15 shows the treatment system 10 pressurized. Once the user determines that the treatment assembly 12 is sufficiently flushed, the flush ports 30c, 30e may be closed to pressurize the treatment assembly 12 with the fluid 76. The syringe 58 may also be removed once the flush port 30c is closed. In the pressurized state, both the valve 28 and the introducer valve 32 are in the sealed state (e.g., as shown in FIGS. 10B and 10D, respectively). The user may confirm the treatment chamber 20 is pressurized by viewing the fluid 76 through the visualization portion 21 of the treatment chamber 20. Once pressurized, the treatment system may receive an endoluminal tool 86 therethrough. The endoluminal tool 86 may be received through the treatment system 10 in a manner similar to a delivery system 90, as discussed with respect to FIGS. 24-28.

FIG. 16 shows the endoluminal tool 86 received through the treatment system 10. The user may introduce the endoluminal tool 86 to the treatment system 10 through the valve 28, the treatment chamber 20, the introducer valve 32, and the introducer sheath 34 to reach the body lumen 69 of the patient. The user may first introduce the endoluminal tool 86 through the valve 28 while the valve 28 is in the sealed state (e.g., as shown in FIG. 10B) such that the valve 28 forms a first hemostatic seal with the endoluminal tool 86. The first hemostatic seal prevents bodily fluids 78, such as blood, from flowing through the valve 28 when the endoluminal tool 86 is inserted therethrough. The endoluminal tool 86 then extends from the valve 28 through the treatment chamber 20 and through the introducer valve 32. Similar to the valve 28, the user may insert the endoluminal tool 86 through the introducer valve 32 when the introducer valve 32 is in the sealed state (e.g., as shown in FIG. 10D) such that the introducer valve 32 forms a second hemostatic seal with the endoluminal tool 86. The second hemostatic seal prevents bodily fluids 78, such as blood, from flowing through the introducer valve 32 when the endoluminal tool 86 is inserted therethrough. The second hemostatic seal is formed while the coupling projection 38 is received within the inner tubular member 40 (e.g., as shown in FIG. 10D).

As shown in FIG. 16, the endoluminal tool 86 may include a retrieval device having a capture mechanism 88 configured to capture the bodily mass 84, such as a thrombus, from the body lumen 69 of the patient. Once captured, the bodily mass 84 may be transferred from the body lumen 69 of the patient and into the introducer sheath 34. Although the bodily mass 84 is shown, a previously implanted endoluminal device (e.g., a heart valve, a stent, or parts thereof, and the like) may be retrieved from the body lumen 69 using a similar retrieval method.

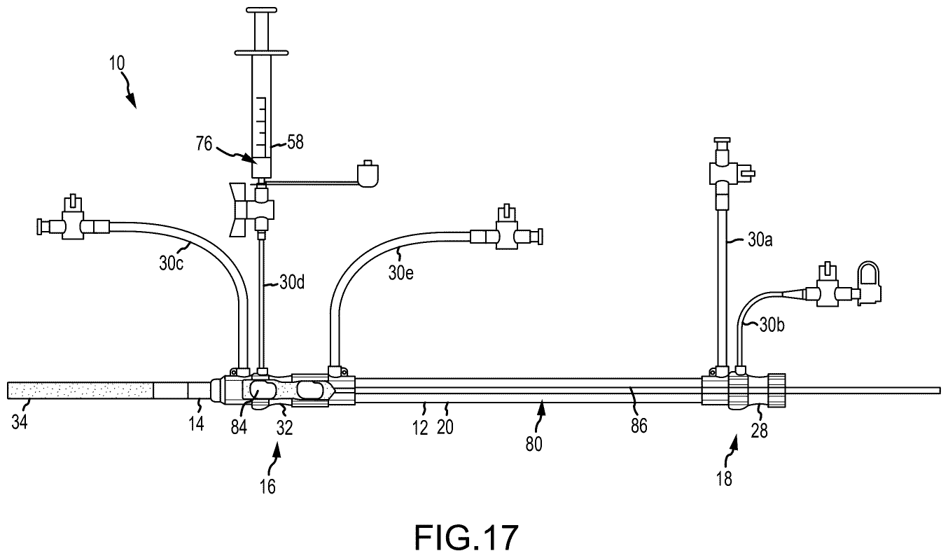

FIGS. 17-21 show the endoluminal tool 86 and the bodily mass 84 traveling through the treatment system 10.

As shown in FIG. 17, while the valve 28 is sealed (e.g., as shown in FIG. 10B), the introducer valve 32 is opened, as shown in FIG. 10C, to withdraw the bodily mass 84 from the introducer sheath 34 through the introducer valve 32 and into the treatment chamber 20 using the endoluminal tool 86. When the introducer valve 32 is open in the unsealed state, the coupling projection 38 remains coupled to the inner tubular member 40 of the introducer valve 32, and the bodily mass 84 is drawn through the inner lumen of the introducer valve 32.

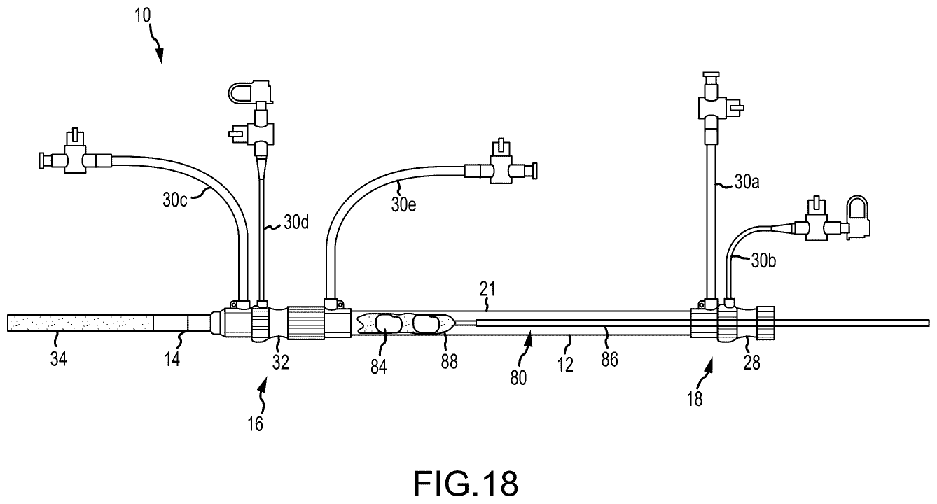

As shown in FIG. 18, once the bodily mass 84 is withdrawn through the introducer valve 32, the introducer valve 32 is sealed such that the bodily mass 84 is isolated from a bodily environment of the patient and is sealed within the treatment chamber 20. The user may visualize the bodily mass 84 and the endoluminal tool 86 through the visualization portion 21 of the treatment chamber 20. In some embodiments, the fluid 76 may be delivered to the treatment chamber 20 once the bodily mass 84 is sealed therein to flush bodily fluids 78 from the treatment chamber 20 such that it is easier for the user to see the bodily mass 84. In some embodiments, and as discussed with respect to FIGS. 8-9, the fluid 76 and bodily fluid 78 may be directed toward and collected within the collection bag 72 of the recirculation loop 64.

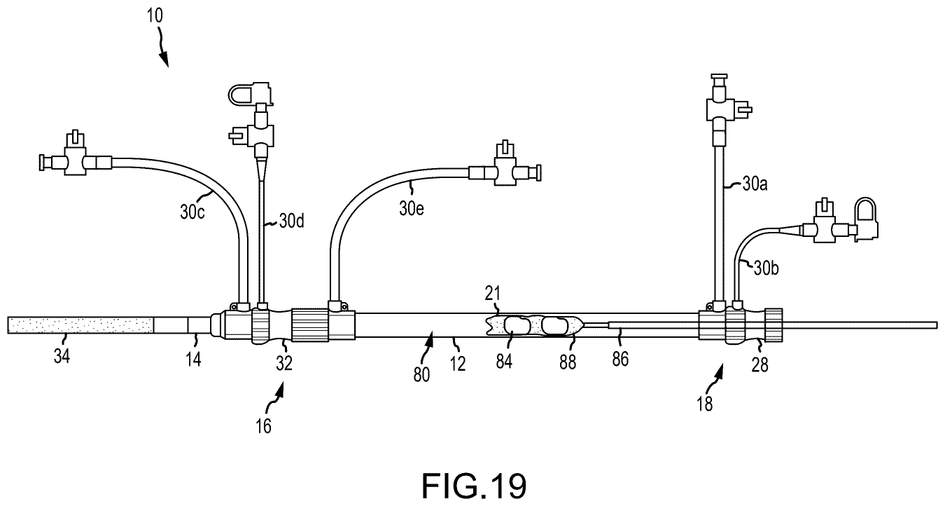

As shown in FIG. 19, the bodily mass 84 may be translated longitudinally along the treatment chamber 20 toward the valve 28. In some embodiments, the bodily mass 84 is moved along the treatment chamber 20 by the user until the bodily mass 84 is positioned within the visualization portion 21. The valve 28 and introducer valve 32 may both be sealed when the bodily mass 84 is moved through the treatment chamber 20 by the endoluminal tool 86 and maintain the first and second hemostatic seals.

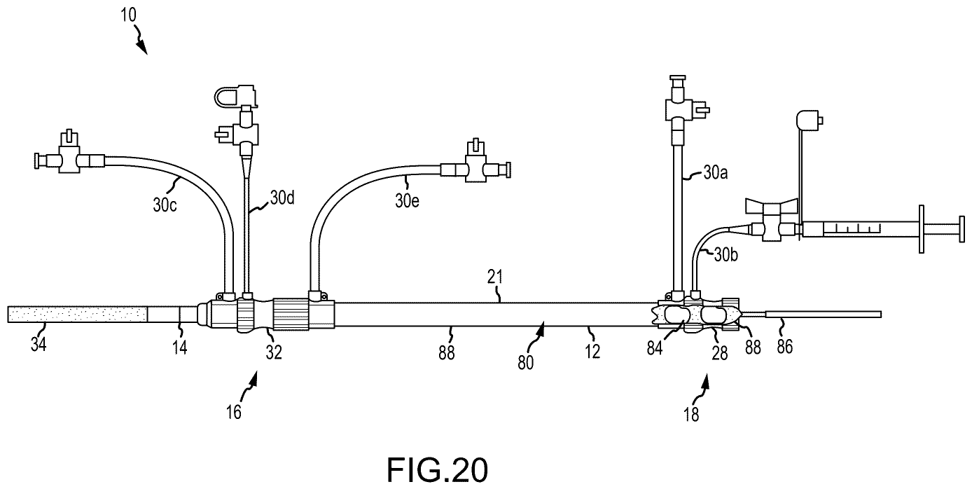

As shown in FIG. 20, while the introducer valve 32 remains sealed, the valve 28 is opened (e.g., as shown in FIG. 10A) to withdraw the bodily mass 84 from the treatment chamber 20 and through the valve 28 using the endoluminal tool 86. The bodily mass 84 may travel through an inner lumen of the valve 28.

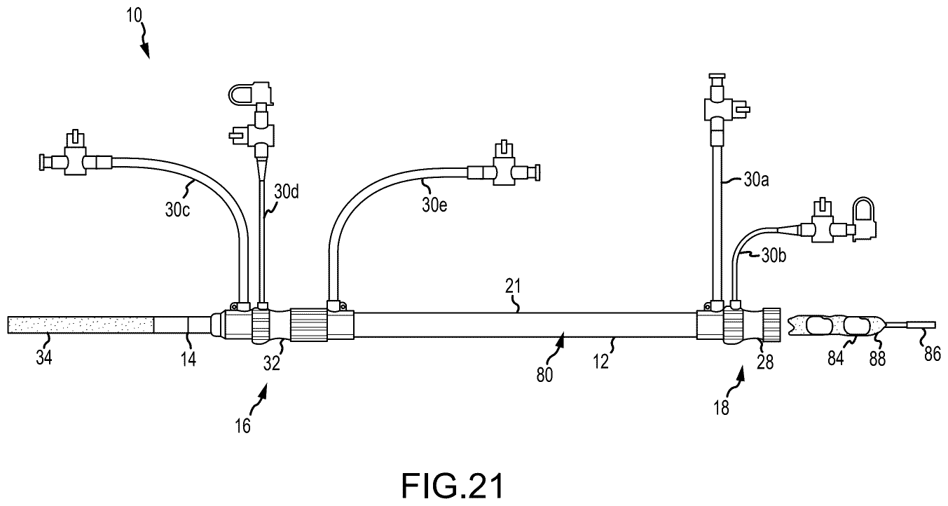

As shown in FIG. 21, once the bodily mass 84 is withdrawn through the valve 28, the valve 28 is closed and sealed such that the bodily mass 84 is isolated outside the treatment system 10. The bodily mass 84 may then be analyzed by the user. In some embodiments, the process of FIGS. 16-21 is repeated to isolate and remove multiple bodily masses 84 from the body lumen 69 of the patient.

As discussed with respect to FIGS. 8-9, the bodily fluid 78 and/or fluid mixture 80 collected from the bodily mass 84 removal may be directed to the recirculation loop 64 such that the fluid mixture 80 enters the treatment chamber 20 and exits the treatment chamber 20 through the recirculation loop 64. In particular, the fluid mixture 80 may enter the recirculation loop 64 proximate to the first end 22 of the treatment chamber 20, travel through the recirculation loop 64, and re-enter the treatment chamber 20 proximate to the second end 24. The recirculation loop 64 may include one or more filters 66 to filter particulates (e.g., portions of a thrombus) out of the fluid mixture 80 prior to reintroduction to the treatment chamber 20 to form a filtered fluid 82. The filtered fluid 82 may then be reintroduced to the patient through the introducer sheath 34. In this manner, blood loss of the patient is minimized during a thrombectomy procedure. Including fluid 76, such as saline, within the filtered fluid 82 may also help enhance fluid recovery for the patient during and/or after the surgical procedure.

After all desired bodily masses 84 are removed from the body lumen 69, the treatment assembly 12 may be decoupled from the introducer assembly 14 by opening the introducer valve 32 and removing the coupling projection 38 from the inner tubular member 40. The introducer assembly 14 is also removed from the body lumen 69 of the patient.

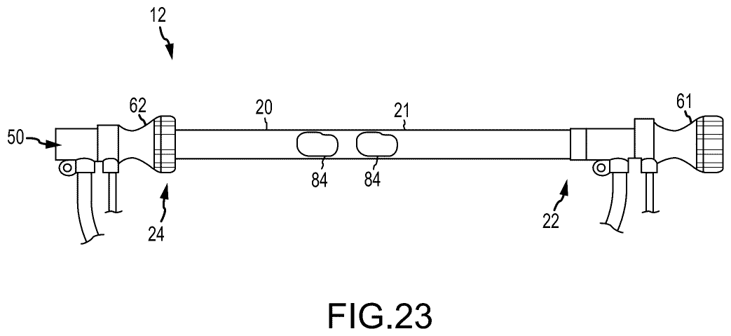

Turning to FIG. 23, in alternative embodiments utilizing the treatment system of FIG. 7, the bodily mass 84 may be removed from the patient and positioned within the treatment chamber 20, similar to the process discussed with respect to FIGS. 15-19. Once the bodily mass 84 is captured within the treatment chamber 20 of the treatment system of FIG. 7, the first valve 61 and the second valve 62 may be closed such that the bodily mass 84 is isolated from a bodily environment of the patient. After sealing both the first and second valves 61, 62, the treatment assembly 12 may be decoupled from the introducer assembly 14 while the bodily mass 84 is contained and sealed within the treatment chamber 20. The bodily mass 84 may then be transported elsewhere for further observation, testing, measurement, analysis, and/or may be disposed.

Turning to FIGS. 24-28, other methods of preparing the treatment system 10 are described. In one embodiment, the treatment system 10 is used to deliver a device to the body lumen 69 of the patient.

FIG. 24 illustrates the treatment system 10, similar to the treatment system 10 described with respect to FIGS. 1-6, prior to introducing a delivery system 90 therein. The treatment assembly 12 may be coupled to the introducer assembly 14 by inserting the coupling projection 38 (FIG. 2) within the inner tubular member 40 (FIGS. 3B-3C) of the introducer assembly 14. Although not shown, the introducer sheath 34 may be placed within the body lumen 69 of the patient (e.g., similar to the process described with respect to FIG. 11).

The delivery system 90 may include an elongate member 92, including but not limited to, a delivery catheter or a push rod. A device 94 may be coupled to a portion the elongate member 92, and the device 94 may be in a delivery configuration. The device 94 includes, but is not limited to, therapeutic devices, monitoring devices, implantable devices, endoprostheses, inflatable balloon catheter including drug coated balloons, heart valves, cardiac repair devices, leadless pacing systems, pellets loaded with an active ingredient, biological therapies, live cell-mediated therapies, electrical monitoring devices, optical monitoring devices, photonic delivery devices, pressure sensors, and/or biomarker sensors.

Prior to introducing the delivery system 90, the valve 28 may be unsealed (e.g., as shown in FIG. 10A). The introducer valve 32 may be sealed with the coupling projection 38 extending therethrough (e.g., as shown in FIG. 10D). By keeping the introducer valve 32 sealed, homeostasis is maintained. The treatment chamber 20 may be filled with ambient air while the introducer valve 32 is sealed, and the valve 28 is unsealed.

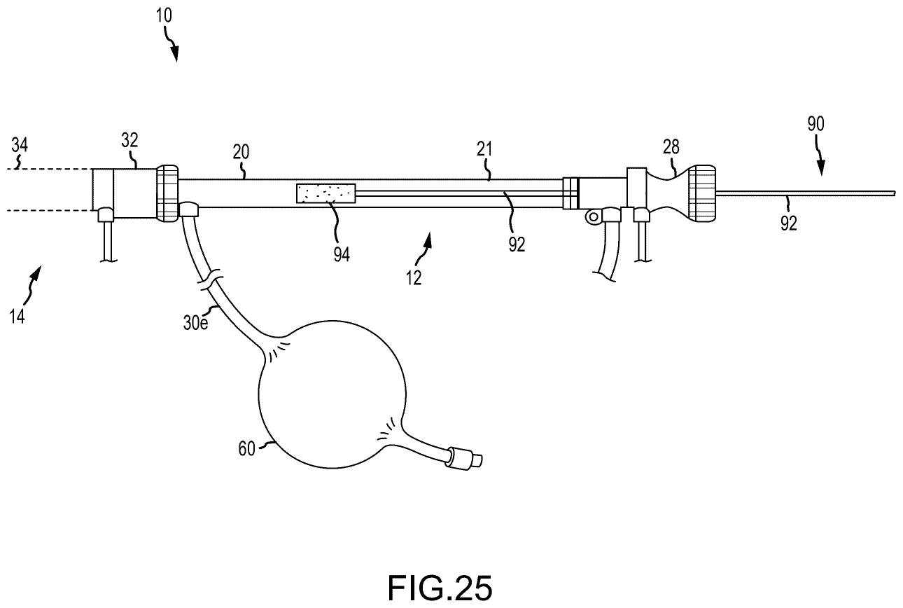

Turning to FIG. 25, the delivery system 90 may be delivered within the treatment chamber 20 through the valve 28 when the valve 28 unsealed. By keeping valve 28 unsealed, mechanical stress on the delivery system 90, including device 94, may be reduced or circumvented upon loading into the treatment chamber 20. The reduction or circumvention of mechanical stress can allow for insertion of delivery systems 90 and/or devices 94, which may include fragile components, without damage from the valves 28.

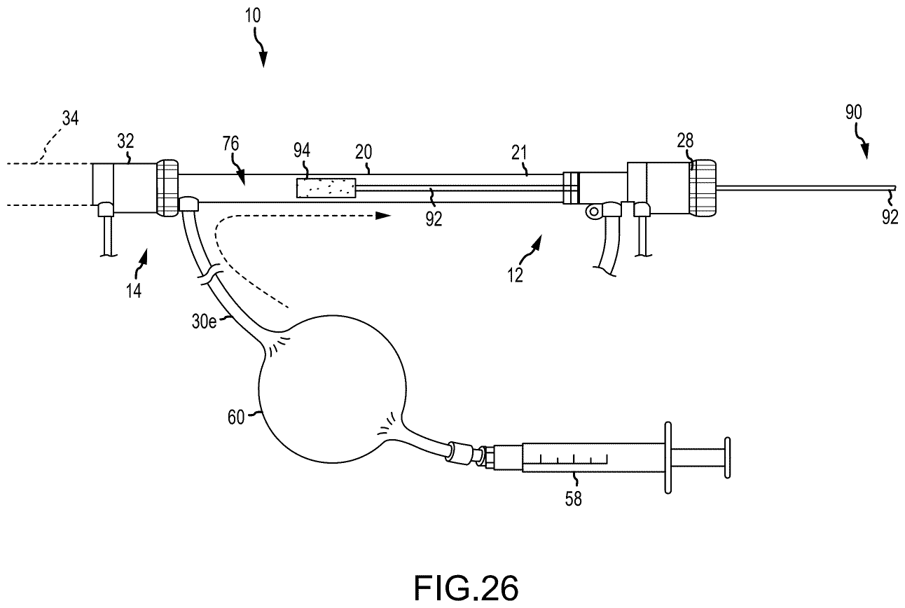

As shown in FIG. 26, when the device 94 is positioned within the treatment chamber 20, the valve 28 may be sealed about the elongate member 92 to form a homeostatic seal with the elongate member 92. The elongate member 92 may be sealed within the valve 28 such that a portion of the elongate member 92 extends within the treatment chamber 20 and another portion of the elongate member 92 extends outside of the treatment chamber 20 (e.g., the elongate member 92 extends through both ends of the valve 28). The introducer valve 32 remains sealed during this process. When both the valve 28 and the introducer valve 32 are sealed, the fluid 76 may be delivered through to the treatment chamber 20 by the syringe 58. The fluid 76 may include saline, or saline supplemented with a therapeutic agent. The therapeutic agent may include agents configured to activate, inhibit, and/or modify one or more mechanical, chemical, or biological properties of the device 94 while the device 94 is sealed within the treatment chamber 20. In some embodiments, several fluid solutions may be delivered to the treatment chamber 20 and flushed therefrom during treatment and preparation of the device 94.

In some embodiments, treatment and preparation of the device 94 includes substantially removing air from the device 94 and the treatment chamber 20 prior to introduction to the body lumen 69. Because the device 94 has been diametrically compacted and collapsed upon itself in the delivery configuration, the device 94 generally defines one or more void spaces, or openings between the diametrically compacted portions of the device 94, which can entrap air. Air may be removed using a fluid media including, but not limited to, carbon dioxide, saline, perfluorocarbon solution, methylene blue, or others. Delivering the fluid media to the treatment chamber 20 forces air out of the chamber lumen 26, and when the device 94 is positioned therein, delivering fluid may help expel entrapped air from the one or more void spaces within the device 94, including entrapped air between a constraint (e.g., a sleeve, not shown) and portions of the device 94 surrounded by the constraint. Entrapped air may be expelled from creases, gaps, and/or folds in the device 94 itself and/or between the device 94 and the constraint. In some embodiments, the delivery system 90 may include one or more vents (not shown) to release air from the treatment system 10, and air may be released in a proximal direction (e.g., toward the valve 28 on the first end 22 of the treatment assembly 12). Release of air may be monitored by viewing the visualization portion 21 of the treatment chamber 20. The volume of the entrapped air may be reduced to 10 μL or less, 5 μL or less, 2 μL or less or other value as desired. Once air has been expelled from the device 94 and the treatment chamber 20, the device 94 is ready for implantation within the body lumen 69.

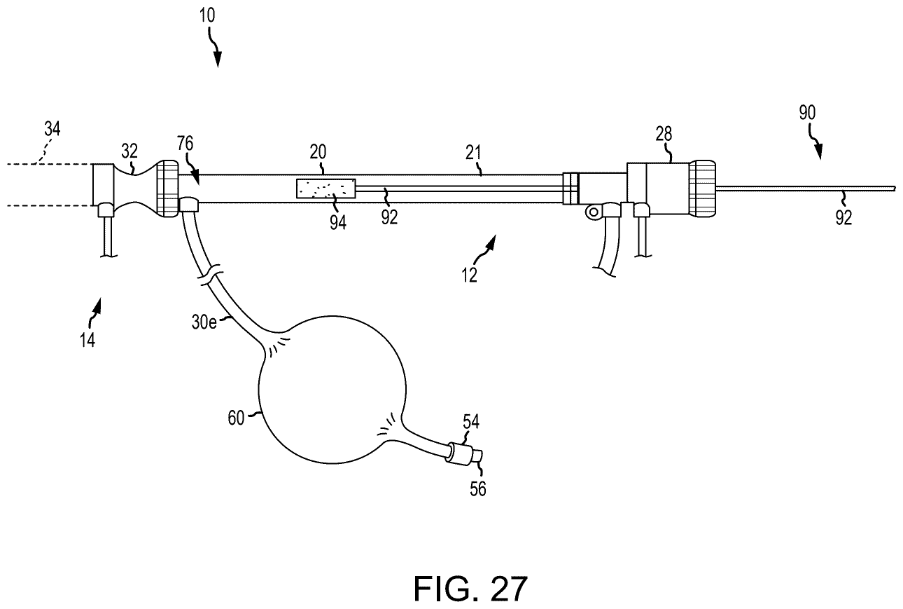

Turning to FIG. 27, after treatment of the device 94 is complete the syringe 58 may be removed and the two-way valve 54 and/or fitting 56 may be sealed (e.g., with a stopcock). The device 94 may then be delivered to the introducer sheath 34 for delivery to the body lumen 89 of the patient. The introducer valve 32 may be unsealed with the coupling projection 38 positioned therein (e.g., as shown in FIG. 10C). When the introducer valve 32 is unsealed, fluid flow between the body lumen 89 and the treatment chamber 20 is enabled. Unsealing the introducer valve 32 to deliver device 94 therethrough helps reduce or circumvent mechanical stress on the delivery system 90, including the device 94, (e.g., as compared to delivering the device 94 through a sealed valve). The valve 28 remains sealed against the elongate member 92 and maintains the homeostatic seal of the treatment system 10.

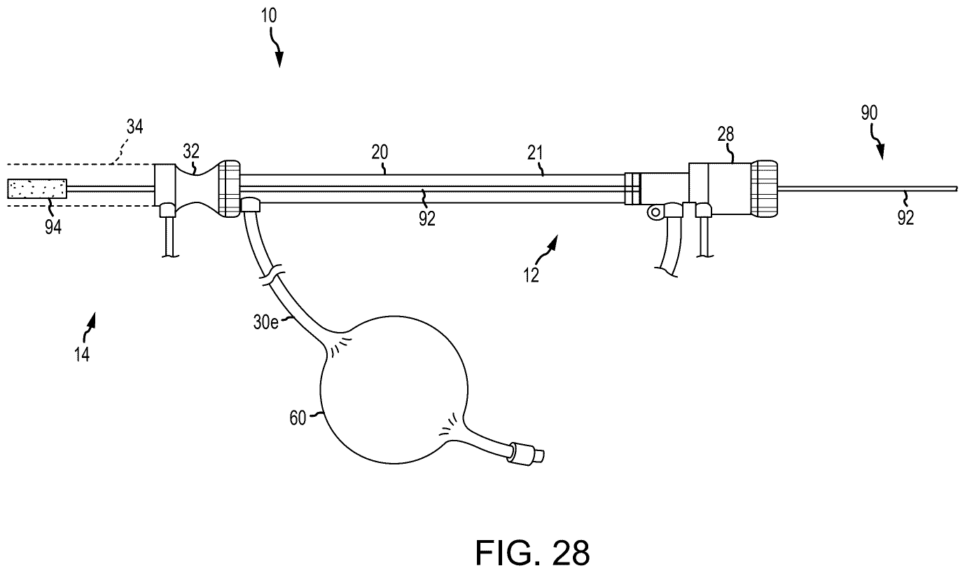

As shown in FIG. 28, the device 94 may then advance through the unsealed introducer valve 32 to enter the introducer sheath 34 for delivery to the body lumen 69. As described, the device 94 is not exposed to mechanical force or stress from the sealed valves, as the valve 28 and introducer valve 32 are opened in sequence to allow the device 94 therethrough. The inclusion of two independently sealed valves allows the system to maintain hemostasis without introducing additional stress to the device 94 during delivery. Such features can allow for insertion of delivery systems 90 and devices 94, which may include fragile components, without damage from the valve 28 or introducer valve 32.

After delivering the device 94, the elongate member 92 may be withdrawn through each of the introducer sheath 34, the introducer valve 32, the treatment chamber 20, and the valve 28. The valve 28 may remain sealed when the elongate member 92 is withdrawn to maintain the homeostatic seal of the treatment system 10.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A treatment system comprising:
a treatment assembly including,
a treatment chamber having a first end and a second end and defining a chamber lumen therebetween,
a valve coupled at the first end of the treatment chamber operable to seal the chamber lumen at the first end, and
a coupling projection at the second end of the treatment chamber; and
an introducer assembly including,
an introducer valve having an inner tubular member, the coupling projection being receivable in the inner tubular member such that the coupling projection is both sealed to the inner tubular member and the inner tubular member remains operable to be transitioned between a sealed state and an unsealed state.

2. The treatment system of claim 1, wherein the introducer valve is operable to seal the second end of the chamber lumen with the coupling projection received in the inner tubular member.

3. The treatment system of claim 1, wherein the treatment assembly includes a collar associated with the coupling projection, the collar operable to engage the introducer valve to limit a length that the coupling projection is inserted into the inner tubular member.

4. The treatment system of claim 1, wherein the treatment assembly includes a gasket associated with the coupling projection, the gasket being operable to seal to the inner tubular member with the coupling projection inserted into the inner tubular member.

5. The treatment system of claim 4, wherein the gasket is inflatable to seal between the inner tubular member and the coupling projection.

6. The treatment system of claim 1, wherein the treatment chamber is longitudinally collapsible and/or diametrically collapsible.

7. The treatment system of claim 6, wherein the treatment chamber includes a plurality of pleats to facilitate longitudinal and/or diametric collapse of the treatment chamber.

8. The treatment system of claim 1, wherein at least a portion of the treatment chamber is at least translucent, and optionally transparent.

9. The treatment system of claim 8, wherein a flush port is coupled to the treatment chamber within the portion of the treatment chamber that is at least translucent.

10. The treatment system of claim 1, wherein the introducer assembly includes an introducer sheath operable to be in fluid communication with to the introducer valve.

11. The treatment system of claim 1, further including a flush port coupled to the treatment chamber, wherein the flush port is in fluid communication with the chamber lumen.

12. The treatment system of claim 11, wherein the flush port is fluidly coupled to a reservoir containing a fluid such that the flush port is operable to move fluid from the reservoir to the chamber lumen.

13. The treatment system of claim 1, further including a recirculation loop fluidly coupled to the treatment chamber such that fluid passing into the treatment chamber is directed out of the treatment chamber through the recirculation loop.

14. The treatment system of claim 1, wherein the recirculation loop further includes a filter.

15. The treatment system of claim 1, wherein the coupling projection is configured to extend into the inner tubular member for less than a full length of the inner tubular member.

16. The treatment system of claim 1, wherein the coupling projection has a length that is less than a length of the inner tubular member.

17. The treatment system of claim 1, wherein the coupling projection includes one or more visual markings demarcating a desired insertion position of the coupling projection into the introducer valve.

18. The treatment system of claim 1, further comprising a collar associated with the second end of the treatment chamber, the collar including a mating feature operable to mate with a complementary feature of the introducer assembly such that the treatment assembly is longitudinally fixed relative to the introducer assembly.

19. The treatment system of claim 18, wherein the mating feature is rotatable to mate with the complementary feature of the introducer assembly.

20. A treatment assembly operable to seal to an introducer valve of an introducer assembly, the treatment assembly comprising:
a treatment chamber having a first end, a second end, and defining a chamber lumen therebetween;
a valve coupled at the first end of the treatment chamber operable to seal the chamber lumen at the first end; and
a coupling projection at the second end of the treatment chamber, the coupling projection being operable to be inserted into the introducer valve such that the introducer valve is sealable to the coupling projection while being operable to seal and unseal an inner lumen of the introducer valve.

21. The treatment assembly of claim 20, further comprising a collar associated with the second end of the treatment chamber, the collar including a mating feature operable to mate with a complementary feature of the introducer assembly such that the treatment assembly is longitudinally fixed relative to the introducer assembly.

22. The treatment assembly of claim 21, wherein the mating feature is rotatable to mate with the complementary feature of the introducer assembly.

23. The treatment assembly of claim 22, further comprising a gasket associated with the coupling projection.

24. The treatment assembly of claim 23, wherein the gasket is operable to inflate.

25. The treatment assembly of claim 20, wherein the treatment assembly includes a collar associated with the coupling projection, the collar operable to engage the introducer valve to limit an insertion distance that the coupling projection is inserted into the inner tubular member.

26. A treatment assembly operable to seal to an introducer valve of an introducer assembly, the treatment assembly comprising:
a treatment chamber having a first end, a second end, and defining a chamber lumen therebetween;
a first valve at the first end of the treatment chamber operable to seal the chamber lumen at the first end;
a second valve at the second end of the treatment chamber operable to seal the chamber lumen at the second end; and
a coupling projection extending from the second valve, the coupling projection being operable to be inserted into the second valve such that the second valve is sealable to the coupling projection while being operable to seal and unseal an inner lumen of the second valve.

27. A method of treatment comprising:
delivering an introducer assembly into a body lumen of a patient, the introducer assembly including an introducer sheath extending into the body lumen and an introducer valve coupled to the introducer sheath and having an inner tubular member;
inserting a coupling projection of a treatment assembly into the inner tubular member of the introducer valve for less than a full length of the inner tubular member, the treatment assembly including a treatment chamber and a valve; and
operating the introducer valve to seal the coupling projection of the treatment assembly to the inner tubular member.

28. A method of treatment comprising:
delivering an introducer assembly into a body lumen of a patient, the introducer assembly including an introducer sheath extending into the body lumen;
coupling a treatment assembly to the introducer assembly, the treatment assembly including a first valve on a first end of the treatment assembly, a second valve on a second end of the treatment assembly and a treatment chamber extending therebetween;
removing a bodily mass from body lumen of the patient;
isolating the bodily mass from a bodily environment of the patient within the treatment chamber; and
removing the treatment assembly from the introducer assembly with the bodily mass contained therein.

29. A method of treatment comprising:
delivering an introducer assembly into a body lumen of a patient, the introducer assembly including an introducer sheath extending into the body lumen and an introducer valve;
coupling a first end of a treatment assembly to the introducer valve, the treatment assembly including a valve on a second end of the treatment assembly and a treatment chamber extending between the first end and the second end;
unsealing the valve of the treatment assembly;
delivering a device into the treatment chamber through the valve of the treatment assembly;
sealing the valve of the treatment assembly;
unsealing the introducer valve; and
advancing the device through the introducer valve for delivery to the body lumen.
